SEQUENCE LISTING (30 sequences)

SEQ ID NO: 1 - protein, 427 aa, Escherichia coli
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFRLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 2 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 3 - DNA, 1284 bp, Escherichia coli
  source 1..1284: mol_type = genomic DNA
atggctgatacaaaagcaaaactcaccctcaacggggatacagctgttgaactggatgtgctgaaaggcacgctgggtcaagatgttattgatatccgtactctcggttcaaaaggtgtgttcacctttgacccaggcttcacttcaaccgcatcctgcgaatctaaaattacttttattgatggtgatgaaggtattttgctgcaccgcggtttcccgatcgatcagctggcgaccgattctaactacctggaagtttgttacatcctgctgaatggtgaaaaaccgactcaggaacagtatgacgaatttaaaactacggtgacccgtcataccatgatccacgagcagattacccgtctgttccatgctttccgtcgcgactcgcatccaatggcagtcatgtgtggtattaccggcgcgctggcggcgttctatcacgactcgctggatgttaacaatcctcgtcaccgtgaaattgccgcgttccgcctgctgtcgaaaatgccgaccatggccgcgatgtgttacaagtattccattggtcagccatttgtttacccgcgcaacgatctctcctacgccggtaacttcctgaatatgatgttctccacgccgtgcgaaccgtatgaagttaatccgattctggaacgtgctatggaccgtattctgatcctgcacgctgaccatgaacagaacgcctctacctccaccgtgcgtaccgctggctcttcgggtgcgaacccgtttgcctgtatcgcagcaggtattgcttcactgtggggacctgcgcacggcggtgctaacgaagcggcgctgaaaatgctggaagaaatcagctccgttaaacacattccggaatttgttcgtcgtgcgaaagacaaaaatgattctttccgcctgatgggcttcggtcaccgcgtgtacaaaaattacgacccgcgcgccaccgtaatgcgtgaaacctgccatgaagtgctgaaagagctgggcacgaaggatgacctgctggaagtggctatggagctggaaaacatcgcgctgaacgacccgtactttatcgagaagaaactgtacccgaacgtcgatttctactctggtatcatcctgaaagcgatgggtattccgtcttccatgttcaccgtcattttcgcaatggcacgtaccgttggctggatcgcccactggagcgaaatgcacagtgacggtatgaagattgcccgtccgcgtcagctgtatacaggatatgaaaaacgcgactttaaaagcgatatcaagcgttaa

SEQ ID NO: 4 - DNA, 2652 bp, Escherichia coli
  source 1..2652: mol_type = genomic DNA
atgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaaccagaaggcctgctgacacaaaacgaagaactgtgggaaccgctctacgcttgctaccagtcacttcaggcgtgtggcatgggtattatcgccaacggcgatctgctcgacaccctgcgccgcgtgaaatgtttcggcgtaccgctggtccgtattgatatccgtcaggagagcacgcgtcataccgaagcgctgggcgagctgacccgctacctcggtatcggcgactacgaaagctggtcagaggccgacaaacaggcgttcctgatccgcgaactgaactccaaacgtccgcttctgccgcgcaactggcaaccaagcgccgaaacgcgcgaagtgctcgatacctgccaggtgattgccgaagcaccgcaaggctccattgccgcctacgtgatctcgatggcgaaaacgccgtccgacgtactggctgtccacctgctgctgaaagaagcgggtatcgggtttgcgatgccggttgctccgctgtttgaaaccctcgatgatctgaacaacgccaacgatgtcatgacccagctgctcaatattgactggtatcgtggcctgattcagggcaaacagatggtgatgattggctattccgactcagcaaaagatgcgggagtgatggcagcttcctgggcgcaatatcaggcacaggatgcattaatcaaaacctgcgaaaaagcgggtattgagctgacgttgttccacggtcgcggcggttccattggtcgcggcggcgcacctgctcatgcggcgctgctgtcacaaccgccaggaagcctgaaaggcggcctgcgcgtaaccgaacagggcgagatgatccgctttaaatatggtctgccagaaatcaccgtcagcagcctgtcgctttataccggggcgattctggaagccaacctgctgccaccgccggagccgaaagagagctggcgtcgcattatggatgaactgtcagtcatctcctgcgatgtctaccgcggctacgtacgtgaaaacaaagattttgtgccttacttccgctccgctacgccggaacaagaactgggcaaactgccgttgggttcacgtccggcgaaacgtcgcccaaccggcggcgtcgagtcactacgcgccattccgtggatcttcgcctggacgcaaaaccgtctgatgctccccgcctggctgggtgcaggtacggcgctgcaaaaagtggtcgaagacggcaaacagagcgagctggaggctatgtgccgcgattggccattcttctcgacgcgtctcggcatgctggagatggtcttcgccaaagcagacctgtggctggcggaatactatgaccaacgcctggtagacaaagcactgtggccgttaggtaaagagttacgcaacctgcaagaagaagacatcaaagtggtgctggcgattgccaacgattcccatctgatggccgatctgccgtggattgcagagtctattcagctacggaatatttacaccgacccgctgaacgtattgcaggccgagttgctgcaccgctcccgccaggcagaaaaagaaggccaggaaccggatcctcgcgtcgaacaagcgttaatggtcactattgccgggattgcggcaggtatgcgtaataccggctaa

SEQ ID NO: 5 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 6 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactgagaaacagct

SEQ ID NO: 7 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 8 - DNA, 141 bp, synthetic construct
  misc_feature 1..141: note = Promoter sequence
  source 1..141: mol_type = other DNA
tgtgagttagctcactcattaggcaccccaggctttacactttatgcttccggctcgtatgttgtgtggaatgcctacaagcatcgtggaggtccgtgactttcacgcatacaacaaacattaaccaaggaggaaacagct

SEQ ID NO: 9 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 10 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 11 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 12 - DNA, 310 bp, synthetic construct
  misc_feature 1..310: note = Promoter sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 13 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 14 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 15 - DNA, 189 bp, synthetic construct
  misc_feature 1..189: note = Promoter sequence
  source 1..189: mol_type = other DNA
gaaaacattcataaattaaatgtgaattgccgcacacattattaaataagatttacaaaatgttcaaaatgacgcatgaaatcacgtttcactttcgaattatgagcgaatatgcgcgatgcctacaagcatcgtggaggtccgtgactttcacgcatacaacaaacattaaccaaggaggaaacagct

SEQ ID NO: 16 - DNA, 239 bp, synthetic construct
  misc_feature 1..239: note = Promoter sequence
  source 1..239: mol_type = other DNA
ccatttagccatagtaaaaacatgaattgtttgatttcgcgcatattcgctcataattcgaaagtgaaacgtgatttcatgcgtcattttgaacattttgtaaatcttatttaataatgtgtgcggcaattcacatttaatttatgaatgttttcttaacatcgcggcatgcctacaagcatcgtggaggtccgtgactttcacgcatacaacaaacattaaccaaggaggaaacagct

SEQ ID NO: 17 - DNA, 300 bp, synthetic construct
  misc_feature 1..300: note = Promoter sequence
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 18 - DNA, 310 bp, synthetic construct
  misc_feature 1..310: note = Promoter sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 19 - DNA, 310 bp, synthetic construct
  misc_feature 1..310: note = Promoter sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 20 - DNA, 310 bp, synthetic construct
  misc_feature 1..310: note = Promoter sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 21 - DNA, 350 bp, synthetic construct
  misc_feature 1..350: note = Promoter sequence
  source 1..350: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 22 - DNA, 310 bp, synthetic construct
  misc_feature 1..310: note = Promoter sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 23 - DNA, 300 bp, synthetic construct
  misc_feature 1..300: note = Promoter sequence
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 24 - DNA, 310 bp, synthetic construct
  misc_feature 1..310: note = Promoter sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 25 - DNA, 189 bp, synthetic construct; identical to SEQ ID NO: 15
  misc_feature 1..189: note = Promoter sequence
  source 1..189: mol_type = other DNA

SEQ ID NO: 26 - DNA, 107 bp, Escherichia coli
  source 1..107: mol_type = other DNA
tgtgagttagctcactcattaggcaccccaggctttacactttatgcttccggctcgtatgttgtgtggaattgtgagcggataacaatttcacacaggaaacagct

SEQ ID NO: 27 - DNA, 300 bp, synthetic construct
  misc_feature 1..300: note = Promoter sequence
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 28 - DNA, 300 bp, synthetic construct
  misc_feature 1..300: note = Promoter sequence
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 29 - DNA, 116 bp, synthetic construct
  misc_feature 1..116: note = Promoter sequence
  source 1..116: mol_type = other DNA
atgcgcaaatttgacggctagctcagtcctaggtacagtgctagcatgcctacaagcatcgtggaggtccgtgactttcacgcatacaacaaacattaaccaaggaggaaacagct

SEQ ID NO: 30 - DNA, 825 bp, Escherichia coli
  source 1..825: mol_type = genomic DNA
atggtcgcacccattcccgcgaaacgcggcagaaaacccgccgttgccaccgcaccagcgactggacaggttcagtctttaacgcgtggcctgaaattactggagtggattgccgaatccaatggcagtgtggcactcacggaactggcgcaacaagccgggttacccaattccacgacccaccgcctgctaaccacgatgcaacagcagggtttcgtgcgtcaggttggcgaactgggacattgggcaatcggcgcacatgcctttatggtcggcagcagctttctccagagccgtaatttgttagcgattgttcaccctatcctgcgcaatctaatggaagagtctggcgaaacggtcaatatggcggtgcttgatcaaagcgatcacgaagcgattattatcgaccaggtacagtgtacgcatctgatgcgaatgtccgcgcctatcggcggtaaattgccgatgcacgcttccggtgcgggtaaagcctttttagcccaactgagcgaagaacaggtgacgaagctgctgcaccgcaaagggttacatgcctatacccacgcaacgctggtgtctcctgtgcatttaaaagaagatctcgcccaaacgcgcaaacggggttattcatttgacgatgaggaacatgcactggggctacgttgccttgcagcgtgtattttcgatgagcaccgtgaaccgtttgccgcaatttctatttccggaccgatttcacgtattaccgatgaccgcgtgaccgagtttggcgcgatggtgattaaagcggcgaaggaagtgacgctggcgtacggtggaatgcgctga

(19) United States
(12) Patent Application Publication
(10) Pub. No.: US 2025/0290078 A1
(43) Pub. Date: Sep. 18, 2025

(54) HMO PRODUCING MICROORGANISM WITH INCREASED ROBUSTNESS TOWARDS GLUCOSE GRADIENTS

(71) Applicant: DSM IP Assets B.V., Maastricht (NL)

(72) Inventors: Greta GECSE, Hørsholm (DK); Margit PEDERSEN, Hørsholm (DK); Ted JOHANSON, Hørsholm (DK)

(21) Appl. No.: 18/860,500

(22) PCT Filed: Apr. 27, 2023
(86) PCT No.: PCT/EP23/61128

§ 371 Date: Oct. 25, 2024

(30) Foreign Application Priority Data
Apr. 29, 2022 (EP) 22170982.7

(51) Int. Cl. C12N 15/70; C12N 9/10; C12N 9/88; C12P 19/18

(52) U.S. Cl. CPC C12N 15/70; C12N 9/1025; C12N 9/1048; C12N 9/88; C12P 19/18; C12Y 203/03001; C12Y 204/01038; C12Y 204/01065; C12Y 204/01069; C12Y 204/01086; C12Y 204/01149; C12Y 204/0115; C12Y 204/01152; C12Y 204/99001; C12Y 204/99004; C12Y 401/01031

(57) ABSTRACT

The present disclosure relates to improved strains for the production of Human Milk Oligosaccharides (HMOs) in large scale. The strains are genetically engineered to produce less acetate and/or ethanol during large-scale fermentation, in particular when encountering gradients with excess carbon source in the fermenter.

### FIELD

The present disclosure relates to the production of Human Milk Oligosaccharides (HMOs) using genetically engineered cells which produce less acetate or ethanol during fermentation. In particular the present disclosure relates to genetically modified cells which overexpress citrate synthase (gltA) in combination with overexpression of phosphoenolpyruvate carboxylase (ppc), and/or decreased expression or an abolished expression of isocitrate lyase regulator (iclR).

### BACKGROUND

The design and construction of bacterial cell factories to produce Human Milk Oligosaccharides (HMOs), is of paramount importance to increase cell robustness for large-scale industrial fermentations for the HMO production of the future.

For HMO production cell lines rational strain engineering principles are commonly applied to single bacterial cells. Such principles usually refer to a) the introduction of a desired biosynthetic pathway to the host, b) the increase of the cellular pools of relevant activated sugars required as donors in the desired reactions, c) the enhancement of lactose import by the native lactose permease LacY and d) the introduction of suitable glycosyltransferases to facilitate the biosynthetic production of oligosaccharides (for review see Bych et al 2019, Current Opinion in Biotechnology 56:130-137).

One of the major challenges in up-scaling aerobic fermentation processes is heterogeneity caused by inefficient mixing. Perfect mixing of volumes large scale bioreactors from approximately 1000 L and more is not feasible, hence, gradients of substrate (Bylund et al. 1998 Bioprocess Engineering, 18(3), 171-180), nutrients, pH and dissolved gases can be formed. In such conditions, the culture is exposed to various harsh conditions and heterogeneity. Glucose gradients were shown trigger E. coli's overflow metabolism at the high glucose concentration zones (Lara et al. 2009 Biotech Bioengineering 104(6), 1153-1161), as well as increase in cells' maintenance requirements eventually leading to performance loss.

Acetate formation is a major by-product of aerobic fermentations in some microorganisms due to glucose overflow metabolism. Elevated acetate concentrations have an inhibitory effect on growth rate and recombinant protein yield and may even lead to failed fermentation batches, resulting in a severe economic loss. Thus, elimination of acetate formation is an important aim towards industrial production. In other organisms such as yeast the overflow metabolism leads to ethanol formation which potentially have similar inhibitory effects as the acetate. For a review of overflow metabolites in different organisms see Taymaz-Nikerel and Lara 2022 Microorganisms 10, 43.

De Maeseneire 2006 Biotechnol Lett 28:1945-1953 describes the effect of acetate reduction and cell density increase in an E. coli cell were citrate synthase (gltA) and phosphoenolpyruvate carboxylase (ppc) is upregulated. The article does not relate to HMO production and does not describe increased robustness to glucose gradients.

Liao et al. 2021 Biotechnology & Biotechnological Equipment, 35, 425-436 describe deletion of iclR and/or arcA in a 2′FL producing strain. Here the iclR deletion markedly reduce the 2′FL production.

In summary, acetate production in large-scale fermentation is a challenge that has been addressed by manipulating the host cell by reducing the formation of pyruvate to acetate by redirecting the carbon flux to the TCA cycle. For HMO production this problem has however not been solved without affecting the HMO yield.

### SUMMARY

The present disclosure relates to a host cell capable of producing HMOs, where the cell has been manipulated to produce less acetate during the fermentation and were the host cell also show increase robustness when subjected to glucose gradients.

One aspect of the disclosure relates to a genetically modified cell capable of producing one or more Human Milk Oligosaccharides (HMOs), wherein said cell comprises the following modifications a) overexpression of citrate synthase (gltA), and b) one or more heterologous nucleic acids encoding one or more glycosyltransferases, and c) at least one biosynthetic pathway for making an activated sugar nucleotide capable of serving as glycosyl-donor for the glycosyl transferase(s) of b). In addition, the genetically modified cell may comprise a further modification d) with at least one of the following modifications: i) overexpression of phosphoenolpyruvate carboxylase (ppc), and/or ii) decreased expression or an abolished expression of isocitrate lyase regulator (iclR).

The genetically modified cell according to the present disclosure can further comprise a promoter element that independently controls the expression of a native or recombinant nucleic acid encoding citrate synthase (gltA) and/or a native or recombinant nucleic acid encoding phosphoenolpyruvate carboxylase (ppc). Where the promoter element controls the native gene, it is stronger than the native promoter and thereby results in overexpression of the native gene.

The genetically modified cell according to the present disclosure can be a microorganism, such as a bacterium or a fungus, wherein said fungus can be selected from a yeast cell, such as of the genera Komagataella, Kluyveromyces, Yarrowia, Pichia, Saccaromyces, Schizosaccharomyces or Hansenula, or from a filamentous fungous of the genera Aspergillus, Fusarium or Trichoderma, and said bacterium can be selected from the exemplified group consisting of Escherichia sp., Bacillus sp., lactobacillus sp., Corynebacterium sp. and Campylobacter sp. Accordingly, the genetically modified cell according to the present disclosure can be E coli.

The genetically modified cell of the present disclosure can be used in the production of an HMO or a mixture of HMOs.

Another aspect of the disclosure relates to a method for producing a human milk oligosaccharide (HMO) comprising the steps of a) providing a genetically modified cell of the present disclosure and b) culturing the cell according to (a) in a suitable cell culture medium to produce said HMO. Preferably, the acetate formation produced by the genetically modified cell of the present disclosure is at least 30% lower as compared to a method where the genetically modified cell does not overexpress citrate synthase (gltA). Even more preferred, the acetate formation produced by the genetically modified cell of the disclosure is at least 40% lower as compared to a method where the genetically modified cell does not overexpress citrate synthase (gltA) and phosphoenolpyruvate carboxylase (ppc) or does not overexpress citrate synthase (gltA) and isocitrate lyase regulator (iclR) expression is abolished.

Various exemplary embodiments and details are described hereinafter, with reference to the figures and sequences when relevant. It should be noted that the figures are only intended to facilitate the description of the embodiments. They are not intended as an exhaustive description of the disclosure or as a limitation on the scope of the disclosure. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

### BRIEF DESCRIPTION OF THE FIGURES

FIG. 1: Acetic acid development in cultures after glucose pulse (example 3). Samples were collected and acetic acid was quantified before pulse addition (t=−5 min), at the addition (t=0) and frequently after until 90 minutes. Experiments with mutants were performed in duplicates (n=2) and triplicates with the control strain (n=3).

FIG. 2: Comparing product formation and growth in fed-batch fermentations of example 2 and 3. The vertical dotted grey lines indicates the window in which the glucose pulse was added to the fermentations in example 3.

FIG. 3: Simplified central carbon metabolism and 2′FL production pathways in E. coli. Gene targets to Increase carbon flux from pyruvate to TCA are marked in bold.

### DETAILED DESCRIPTION

The present disclosure approaches the biotechnological challenges of in vivo HMO production, in particular large-scale production where heterogeneity arises in the fermentation broth due to inefficient mixing which may result in overflow metabolism in the cells. The overflow metabolism in many bacteria cause high acetate formation and affect product yields. In many yeasts the overflow metabolism causes high ethanol formation which equally affect product yields, and in some species formation of both acetate and ethanol is observed (Taymaz-Nikerel and Lara 2022 Microorganisms 10, 43).

In other words, a genetically modified cell covered by the present disclosure provides a host cell which produces less acetate or ethanol during fermentation, and which show increased robustness towards glucose gradients.

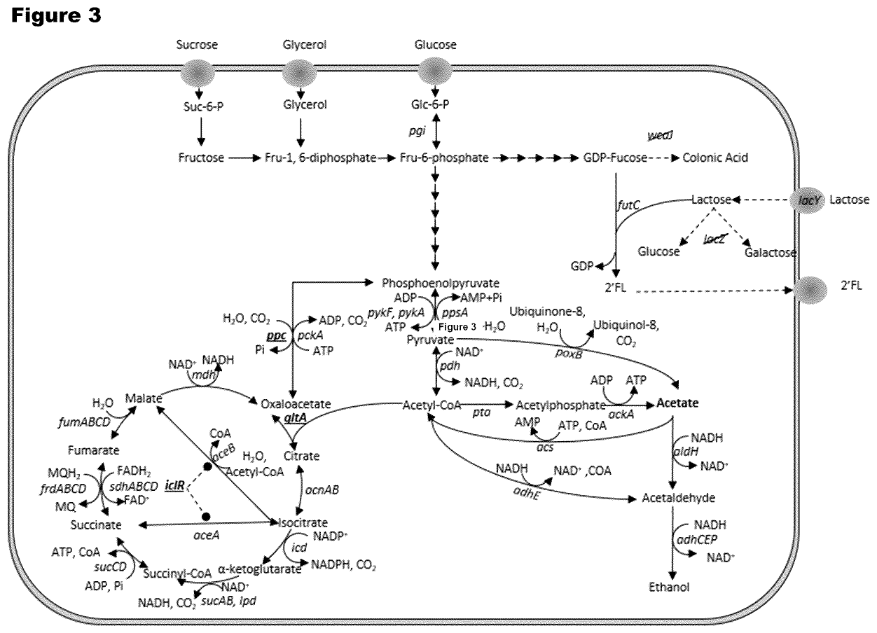

In the examples of this application genes connected to the TCA cycle have been investigated for their ability to reduce acetic acid/acetate formation during fermentation without decreasing the HMO production as well as for their ability in withstanding glucose pulses. Manipulation of one or more of the following genes a) citrate synthase (gltA) and b) phosphoenolpyruvate carboxylase (ppc) and c) isocitrate lyase regulator (iclR) were found to have an effect on acetate formation, either alone or in combination. FIG. 3 illustrates the pathways affected in the modified cell of the present disclosure, where the cell is a 2′FL producing cell.

In one embodiment of the present disclosure the genetically modified cell is capable of producing one or more HMOs and which overexpress citrate synthase (gltA), preferably in combination with overexpression of phosphoenolpyruvate carboxylase (ppc). As shown in example 1 this leads to a more than 40% acetate reduction compared to the same strain without these modifications (i.e. which does not overexpress glta and pcc), while the HMO production is not significantly decreased. Furthermore, this strain is not affected by glucose gradients introduced into the fermentation via a glucose pulse (example 4).

In another embodiment of the present disclosure the genetically modified cell is capable of producing one or more HMOs and overexpress citrate synthase (gltA) and preferably the isocitrate lyase regulator (iclR) has also been mutated to prevent expression of functional iclR. As shown in example 1 this leads to a more than 70% acetate reduction compared to the same strain without these modifications (i.e. which does not overexpress glta and does not contain an iclR deletion), while the HMO production is not decreased and maybe even increased. Furthermore, this strain is not affected by glucose gradients introduced into the fermentation via a glucose pulse (example 4).

In a further embodiment of the present disclosure the genetically modified cell is capable of producing one or more HMOs and overexpress citrate synthase (gltA) and phosphoenolpyruvate carboxylase (ppc) and the isocitrate lyase regulator (iclR) has been mutated to prevent expression of functional iclR.

Citrate Synthase (gltA)

Citrate synthase is an enzyme belonging to E.C. 2.3.3.1 and exists in nearly all living cells. Citrate synthase is in the first step of the citric acid cycle where it catalyzes the condensation reaction of the two-carbon acetate residue from acetyl coenzyme A and a molecule of four-carbon oxaloacetate to form the six-carbon citrate. Citrate synthase is encoded by the gltA gene in E. coli and Corynebacterium, but can have different names and isoforms in other organisms, such as CIT1, CIT2 and CIT 3 in S. cerevisiae or citA, citZ, ctsA or mmgD in Bacillus. In the context of the present disclosure gltA refers to a gene encoding citrate synthase independent of the strain encoding it.

In one embodiment of the present disclosure citrate synthase can for example be overexpressed by placing the native gltA gene under control of a promoter that is stronger than the native promoter, or by inserting a recombinant nucleic acid encoding a citrate synthase into the cell. In a preferred embodiment the promoter controlling the expression of the citrate synthase is selected from table 10.

In one embodiment the nucleic acid encoding the citrate synthase comprises or consists of the amino acid sequence of SEQ ID NO: 1 or an amino acid sequence with at least 80%, such as at least 85%, such as at least 90%, such as at least 95%, or such as at least 99% identity to SEQ ID NO: 1.

The recombinant nucleic acid encoding a citrate synthase may be episomally expressed, e.g. plasmid borne or integrated into the genome of the genetically modified cell. For large scale production integration into the genome is preferred in order to avoid selection pressure to maintain the plasmid in the cell.

Phosphoenolpyruvate Carboxylase (ppc)

Phosphoenolpyruvate carboxylase (also known as PEP carboxylase, PEPCase, or PEPC) is an enzyme belonging to EC 4.1.1.31 which is found in plants and bacteria. Phosphoenolpyruvate carboxylase catalyzes the formation of oxaloacetate from phosphoenolpyruvate (PEP) by fixing one carbon in the forms of either carbon dioxide or bicarbonate. Phosphoenolpyruvate carboxylase is encoded by the ppc gene in E. coli, Corynebacterium and Bacillus and ppc1 in S. cerevisiae. In the context of the present disclosure ppc refers to a gene encoding Phosphoenolpyruvate carboxylase independent of the strain encoding it.

In one embodiment of the present disclosure phosphoenolpyruvate carboxylase can for example be overexpressed by placing the native ppc gene under control of a promoter that is stronger than the native promoter, or by inserting a recombinant nucleic acid encoding a Phosphoenolpyruvate carboxylase into the cell. In a preferred embodiment the promoter controlling the expression of the citrate synthase is selected from table 10.

In one embodiment the nucleic acid encoding the Phosphoenolpyruvate carboxylase comprises or consists of the amino acid sequence of SEQ ID NO: 2 or an amino acid sequence with at least 80%, such as at least 85%, such as at least 90%, such as at least 95%, or such as at least 99% identity to SEQ ID NO: 2.

The recombinant nucleic acid encoding a Phosphoenolpyruvate carboxylase may be plasmid borne or integrated into the genome of the genetically modified cell. For large scale production integration into the genome is preferred in order to avoid selection pressure to maintain the plasmid in the cell.

Isocitrate Lyase Regulator Family (iclR)

The isocitrate lyase regulator family or IclR family is widely known in microbial organisms such as gram-positive and Alpha-, Beta- and Gamma-proteobacteria and Archaea, often under different names than IclR (Molina-Henares et al 2006 FEMS Microbiol Rev 30: 157-186), but is also found in certain fungi and plants. In E. coli, IclR is a DNA-binding transcriptional regulator that regulates gene expression of aceBAK operon which encodes isocitrate lyase (aceB), malate synthase (aceA) and isocitrate dehydrogenase kinase/phosphorylase (aceK) in the glyoxylate bypass. Inactivation of IclR activates glyoxylate bypass pathway. In the context of the present disclosure the term Isocitrate lyase regulator or iclR is meant to encompass members of the isocitrate lyase regulator family and not only the E. coli IclR. IclR family members are well characterized across species, and can easily be identified by the skilled person in the art. In E. coli one example of the IclR protein is NCBI accession nr. AAC76988.2.

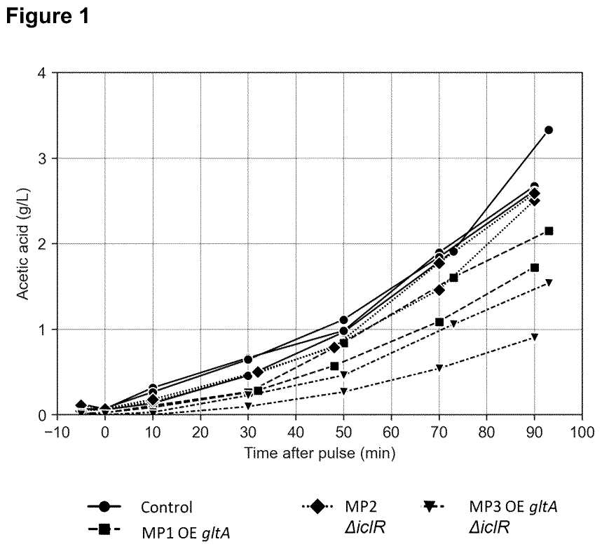

In the context of the present disclosure the function of the native isocitrate lyase regulator family member has been decreased or completely abolished. The function can be lost or abolished by deleting the iclR gene or rendering it dysfunctional e.g. by introducing a nonsense mutation resulting in a premature stop codon rendering the regulator inactive. Preferably the iclR gene is deleted in the host cell. An exemplary iclR gene sequence is provided as SEQ ID NO: 30. Deletion of functional homologues of SEQ ID NO: 30 is encompassed by the present disclosure. A functional homologue may not share a high degree of sequence similarity with SEQ ID NO: 30 but its inactivation does activate glyoxylate bypass pathway (FIG. 1). In one embodiment the iclR gene sequence to be deleted in a cell is at least 50% identical to SEQ ID NO: 30, such as at least 60%, such as at least 70%, such as at least 80%, such as at least 85%, such as at least 90%, such as at least 95%, or such as at least 99% identical to SEQ ID NO: 30.

Reduced function of IclR can be achieved by introducing one or more missense mutation in the iclR gene that results in a change in the amino acid sequence that reduces its ability of the regulator to bind to the aceBAK operon. The function may also be reduced by reducing the expression level of the iclR gene, e.g. by substituting the native promoter with a weaker promoter or a promoter which is only induced under certain conditions. Preferably the functionality or expression level of the IclR is reduced by at least 25%, such as at least 50%, such as at least 75%, such as at least 85%, such as at least 90%.

### Human Milk Oligosaccharide (HMO)

In the context of the disclosure, the term “oligosaccharide” means a saccharide polymer containing a number of monosaccharide units. In some embodiments, preferred oligosaccharides are saccharide polymers consisting of three, four, five or six monosaccharide units, i.e., trisaccharides, tetrasaccharides, pentasaccharides or hexasaccharides. Most preferred are trisaccharides or tetrasaccharides. Preferable oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl unit, and this core structure can be substituted by an alpha-L-fucopyranosyl and/or an alpha-N-acetyl-neuraminyl (sialyl) moiety. HMO structures are e.g., disclosed by Xi Chen in Chapter 4 of Advances in Carbohydrate Chemistry and Biochemistry 2015 vol 72.

In the context of the present disclosure, lactose is not regarded as an HMO species.

HMOs can be non-acidic (or neutral) or acidic. Neutral HMOs are devoid of a sialyl residue and acidic have at least one sialyl residue in their structure. The non-acidic (or neutral) HMOs can be fucosylated or non-fucosylated. Examples of such neutral non-fucosylated HMOs include lacto-N-triose 2 (LNT-2) lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-1), lacto-N-difucohexaose I (LNDFH-I), 3-fucosyllactose (3′-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-1), sialyl-lacto-N-neohexaose II (SLNH-II) and disialyl-lacto-N-tetraose (DSLNT). See for example Bych et al 2019 Current Opinion in Biotechnology 56:130-137 for a review on HMO production.

In one or more preferred embodiment(s), the one or more produced HMO is selected from the group consisting of LNT-II, pLNnH, LNT and LNnT, LNFP-I, LNFP-II, LNFP-III, LNFP-V, LNFP-VI, LNDFH-1, LNDFH-II, LNDFH-III, 2′FL, DFL, 3FL, LST-a, 3′SL, 6′SL, LST-b, LST-c, FSL, FLST-a, DSLNT, LNnH and LNH.

In one embodiment the one or more HMO produced by the herein disclosed method and/or genetically engineered cell is an HMO that comprises an LNT-II core, such as LNT and/or LNnT, pLNnH and/or LNFP-I. Preferably, the produced HMO is LNT and/or LNnT.

In one embodiment the one or more HMO produced by the herein disclosed method and/or genetically engineered cell is a fucosylated HMO, such as 2′FL, 3FL, DFL, FSL, LNFP-I, LNFP-II, LNFP-III, LNFP-V, LNFP-VI, LNDFH-1, LNDFH-II or LNDFH-III. Preferably, the produced HMO is a fucosylactose, such as 2′FL, 3FL or DFL. Even more preferably the produced the HMO is 2′FL.

In one embodiment the one or more HMO produced by the herein disclosed method and/or genetically engineered cell is a sialylated HMO, such as 3′SL, 6′SL, LST-a, LST-b, LST-c, FSL or DSLNT. Preferably, the produced HMO is sialyllactose, such as 3′SL or 6′SL.

### An Acceptor Oligosaccharide

A genetically modified cell according to the present disclosure comprises at least one recombinant nucleic acid sequence encoding a glycosyltransferase activity capable of transferring glycosyl moiety from an activated sugar to a galactose, glucose or N-acetylglucosamine moiety in an acceptor oligosaccharide.

In the context of the present disclosure, an acceptor oligosaccharide is an oligosaccharide that can act as a substrate for a glycosyltransferase capable of transferring a glycosyl moiety from a glycosyl-donor to the acceptor oligosaccharide. The glycosyl-donor is preferably a nucleotide-activated sugar as described in the section on “glycosyltransferases”. The acceptor oligosaccharide can be a precursor for making a more complex HMO (composed of 4 monosaccharides or more). The acceptor oligosaccharide can therefore also be termed precursor molecule.

The acceptor oligosaccharide can be either an intermediate product of the present fermentation process, an end-product of a separate fermentation process employing a separate genetically modified cell, or an enzymatically or chemically produced molecule.

In the present context, said acceptor oligosaccharide for the glycosyltransferase can be for example be lactose, lacto-N-triose II (LNT-II), lacto-N-tetraose (LNT), lacto-N-neotetraose (LNnT), 2′-fucosyllactose (2′FL), 3-fucosyllactose (3FL), 3′-sialyllactose (3′SL) or 6′-salyllactose (6′SL). In a preferred embodiment the acceptor molecule is lactose and is fed to the genetically modified cell which is capable of producing an HMO which in turn may also act as an acceptor molecule inside the cell, e.g. LNT-II acts as acceptor molecule/substrate for a beta-1,3-glycosyltransferase to generate LNT, which then in turn can act as an acceptor molecule for a fucosyltransferase generating for example LNFP-I.

### Glycosyltransferases

The genetically modified cell according to the present disclosure comprises at least one recombinant nucleic acid sequence encoding at least one glycosyltransferase capable of transferring a glycosyl residue from a glycosyl-donor to an acceptor oligosaccharide to synthesize a human milk oligosaccharide product. The nucleic acid sequence encoding the one or more expressed glycosyltransferase(s) may be integrated into the genome (by chromosomal integration) of the genetically engineered cell, or alternatively, it may be comprised in a plasmid and expressed as plasmid-borne, as described in the present disclosure.

The genetically modified cell according to the present disclosure may comprise at least two recombinant nucleic acid sequences encoding two different glycosyltransferases capable of transferring a glycosyl residue from a glycosyl-donor to an acceptor oligosaccharide.

The one or more glycosyltransferase is preferably selected from the group of enzymes having the activity of an α-1,2-fucosyltransferase, α-1,3-fucosyltransferase, α-1,3/4-fucosyltransferase, α-1,4-fucosyltransferase α-2,3-sialyltransferase, α-2,6-sialyltransferase, β-1,3-N-acetylglucosaminyltransferase, β-1,6-N-acetylglucosaminyltransferase, β-1,3-galactosyltransferase and β-1,4-galactosyltransferase, described in more detail below.

Beta-1,3-N-acetyl-glucosaminyltransferase A β-1,3-N-acetyl-glucosaminyltransferase is any protein which comprises the ability of transferring the N-acetyl-glucosamine of UDP-N-acetyl-glucosamine to lactose or another acceptor molecule, in a beta-1,3-linkage. Preferably, a β-1,3-N-acetyl-glucosaminyltransferase used herein does not originate in the species of the genetically engineered cell i.e. the gene encoding the β-1,3-galactosyltransferase is of heterologous origin. Non-limiting examples of β-1,3-N-acetyl-glucosaminyltransferase are given in table 1. β-1,3-N-acetyl-glucosaminyltransferase variants may also be useful, preferably such variants are at least 80%, such as at least 85%, such as at least 90, such as at least 95% identical to one of the β-1,3-N-acetyl-glucosaminyltransferase in table 1.

TABLE 1
List of β-1,3-N-acetyl-glucosaminyltransferase

| Protein | | | |
|---|---|---|---|
| Name | GenBank ID | origin | HMO product |
| lgtA | WP_033911473.1 | Neisseria | LNT-II, LNT, LNnT, LNFP-I, LNFP-II, |
| | WP_002248149 | meningitidis | LNFP-III, LNFP-V, LNFP-VI, LNDFH-I, |
| | AAF42258.1 | | LNDFH-II, LNDFH-III, pLNH, F-pLNH I, |
| | ELK60643.1 | | pLNnH, LST a, LST b, LST c, DSLNT |
| lgtA | AAK70338.1 | Neisseria | LNT-II, LNT, LNnT, LNFP-I, LNFP-II, |
| | ACF31229.1 | gonorrhoeae | LNFP-III, LNFP-V, LNFP-VI, LNDFH-I, |
| | | | LNDFH-II, LNDFH-III, pLNH, F-pLNH I, |
| | | | pLNnH |
| lgtA | AAK02595.1 | Pasteurella | LNT-II, LNT, LNnT, LNFP-I, LNFP-II, |
| | | multocida | LNFP-III, LNFP-V, LNFP-VI, LNDFH-I, |
| | | | LNDFH-II, LNDFH-III, pLNH, F-pLNH I, |
| | | | pLNnH |
| lgtA | EEZ72046.1 | Neisseria | LNT-II, LNT, LNnT, LNFP-I, LNFP-II, |
| | | cinerea | LNFP-III, LNFP-V, LNFP-VI, LNDFH-I, |
| | | | LNDFH-II, LNDFH-III, pLNH, F-pLNH I, |
| | | | pLNnH |
| HD0466 | WP_010944479.1 | Haemophilus | LNT-II, LNT, LNnT, LNFP-I, LNFP-II, |
| | | ducreyi | LNFP-III, LNFP-V, LNFP-VI, LNDFH-I, |
| | | | LNDFH-II, LNDFH-III, pLNH, F-pLNH I, |
| | | | pLNnH, LST a, LST b, LST c, DSLNT |
| PmnagT | WP_014390683.1 | Pasteurella | LNT-II, LNT, LNnT, LNFP-I, LNFP-II, |
| | | multocida | LNFP-III, LNFP-V, LNFP-VI, LNDFH-I, |
| | | | LNDFH-II, LNDFH-III, pLNH, F-pLNH I, |
| | | | pLNnH, LST a, LST b, LST c, DSLNT |

### Heterologous β-1,6-N-Acetylglucosaminyltransferase

A heterologous β-1,6-N-acetyl-glucosaminyl-transferase is any protein which comprises the ability of transferring the N-acetyl-glucosamine of UDP-N-acetyl-glucosamine to an acceptor molecule, in a beta-1,6-linkage. A β-1,6-N-acetyl-glucosaminyl-transferase used herein does not originate in the species of the genetically engineered cell i.e., the gene encoding the β-1,6-galactosyltransferase is of heterologous origin. An example of a β-1,6-N-acetyl-glucosaminyl-transferase is Csp2 from Chryseobacterium sp. KBWO3 (NOBI accession Nr. WP_22844786.1) or a variant thereof which for example can produce LNH or LNnH.

### β-1,3-Galactosyltransferase

A β-1,3-Galactosyltransferase is any protein that comprises the ability of transferring the galactose of UDP-Galactose to a N-acetyl-glucosaminyl moiety to an acceptor molecule in a beta-1,3-linkage. Preferably, a β-1,3-galactosyltransferase used herein does not originate in the species of the genetically engineered cell i.e., the gene encoding the β-1,3-galactosyltransferase is of heterologous origin. Non-limiting examples of β-1,3-galactosyltransferases are given in table 2. β-1,3-galactosyltransferases variants may also be useful, preferably such variants are at least 80%, such as at least 85%, such as at least 90, such as at least 95% identical to one of the β-1,3-galactosyltransferases in table 2.

TABLE 2
List of beta-1,3-glycosyltransferases

| Protein | | | HMO |
|---|---|---|---|
| Name | GenBank ID | Origin | product |
| WbgO | WP_000582563.1 | E. coli 055:H7 | LNT |
| cpslBJ | AB050723.1 | Streptococcus agalactiae | LNnT |
| | | type lb Ol2 | |
| Jhp0563 | AEZ55696.1 | Helicobacter pylori | LNnT |
| GalTK | WP_111735921.1 | Helicobacter pylori | LNT |
| | | ATCC 43504 | |
| CvB3GalT | WP_080969100.1 | Chromobacterium violaceum | LNT |

### β-1,4-Galactosyltransferase

A β-1,4-Galactosyltransferase is any protein that comprises the ability of transferring the galactose of UDP-Galactose to a N-acetyl-glucosaminyl moiety. Preferably, a β-1,4-galactosyltransferase used herein does not originate in the species of the genetically engineered cell i.e., the gene encoding the β-1,4-galactosyltransferase is of heterologous origin. Non-limiting examples of β-1,4-galactosyltransferases are given in table 3. β-1,4-galactosyltransferases variants may also be useful, preferably such variants are at least 80%, such as at least 85%, such as at least 90, such as at least 95% identical to one of the β-1,4-galactosyltransferases in table 3.

TABLE 3
List of beta-1,4-glycosyltransferases

| Protein | | | HMO |
|---|---|---|---|
| Name | GenBank ID | Origin | product |
| GalT | WP_001262061.1 | Helicobacter pylori | LNnT |
| LgtB | AAF42257.1 | Neisseria meningitidis | LNnT |
| | | MC58 | |

### Alpha-1,2-Fucosyltransferase

An α-1,2-fucosyltransferase is a protein that comprises the ability to catalyze the transfer of fucose from a donor substrate, for example, GDP-fucose, to an acceptor molecule in an alpha-1,2-linkage. Preferably, an alpha-1,2-fucosyltransferase used herein does not originate in the species of the genetically engineered cell i.e., the gene encoding the alpha-1, 2-fucosyltransferase is of heterologous origin. Non-limiting examples of alpha-1,2-fucosyltransferase are given in table 4. Alpha-1,2-fucosyltransferase variants may also be useful, preferably such variants are at least 80%, such as at least 85%, such as at least 90, such as at least 95% identical to one of the alpha-1,2-fucosyltransferase in table 4.

TABLE 4
List of α-1,2-fucosyltransferase

| Protein Name | GenBank ID | Origin | HMO product |
|---|---|---|---|
| FutC | WP_080473865.1 | Helicobacter pylori | 2′FL, DFL, |
| | | ATCC 26695 | LNFP-I |
| wbgL | ADN43847 | E. coli O126 | DFL |
| Smob | WP_126455392.1 | Sulfuriflexus mobilis | LNFP-I |
| FucT2_HpUA802 | AAC99764.1 | Helicobacter pylori | 2′FL, LNFP-I, |
| | | | LNDFH-I |
| FucT2 EcO126t | ABE98421.1 | Escherichia coli | 2′FL, LNFP-I, |
| | | | LNDFH-I |
| FucT2_Hm12198 | CBG40460.1 | Helicobacter mustelae | 2′FL, LNFP-I, |
| | | 12198 | LNDFH-I |
| FucT2_Pm9515 | ABM71599.1 | Brochlorococcus marinus | 2′FL, LNFP-I, |
| | | str. MIT 9515 | LNDFH-I |
| FucT2_HpF57 | BAJ59215.1 | Helicobacter pylori | 2′FL, LNDFH-I, |
| | | F57 | LNDFH-I |
| FucT54 | ADE13114.1 | Sideroxydans lithotrophicus | 2′FL, LNFP-I, |
| | | ES-11 | LNDFH-I |
| Mtun | WP_031437198.1 | Methylobacter | 2′FL, LNFP-I, |
| | | tundripaludum | LNDFH-I |

### Alpha-1,3-Fucosyltranferase

An alpha-1,3-fucosyltranferase refer to a glycosyltransferase that catalyzes the transfer of fucose from a donor substrate for example, GP-fucose, to an acceptor molecule in an alpha-1,3-linkage. Preferably, an alpha-H,3-fucosyltransferase used herein does not originate in the species of the genetically engineered cell i.e., the gene encoding the alpha-1,3-fucosyltransferase is of heterologous origin. Non-limiting examples of alpha-1,3-fucosyltransferase are given in table 5. Alpha-1,3-fucosyltransferase variants may also be useful, preferably such variants are at least 80%, such as at least 85%, such as at least 90, such as at least 95% identical to one of the alpha-1,3-fucosyltransferase in table 5.

TABLE 5
List of α-1,3-fucosyltransferase

| Protein Name | GenBank ID | Origin | HMO product |
|---|---|---|---|
| FucT | AAB81031.1 | Helicobacter pylori | 3FL, DFL, LNFP- |
| | | NCTC 11639 | III, LNDFH-III |
| FutA | WP_000487428.1 | Helicobacter pylori | 3FL, DFL, LNFP- |
| | | ATCC 26695 | VI, LNDFH-III |
| CafC | WP_007483358.1 | Bacteroides nordii | 3FL, LNDFH-III |
| FucT6_3_Bf | CAH09151.1 | Bacteroides fragilis | 3FL, LNFP-V, |
| | | NCTC 9343 | LNDFH-II |
| FucT7_3_Bf | CAH09495.1 | Bacteroides fragilis | 3FL, LNFP-V, |
| | | NCTC 9343 | LNDFH-II |
| FucT_3_Am | ACD04596.1 | Akkermansia muciniphila | 3FL, LNFP-V, |
| | | ATCC BAA-835 | LNDFH-II |
| MAMA_R764 | AGC02224.1 | Acanthamoeba polyphaga | 3FL, LNFP-V, |
| | | moumouvirus | LNDFH-II |
| Mg791 | AEQ33441.1 | Megavirus chiliensis | 3FL, LNFP-V, |
| | | | LNDFH-II |
| Moumou_00703 | YP_007354660.1 | Acanthamoeba polyphaga | 3FL, LNFP-V, |
| | | moumouvirus | LNDFH-II |

### Alpha-1,3/4-Fucosyltransferase

An alpha-1,3/4-fucosyltransferase refer to a glycosyltransferase that catalyzes the transfer of fucose from a donor substrate for example, GDP-fucose, to an acceptor molecule in an alpha-1,3- or alpha 1,4-linkage. Preferably, an alpha-1,3/4-fucosyltransferase used herein does not originate in the species of the genetically engineered cell i.e., the gene encoding the alpha-1,3/4-fucosyltransferase is of heterologous origin. Non-limiting examples of alpha-1,3/4-fucosyltransferase are given in table 6. alpha-1,3/4-fucosyltransferase variants may also be useful, preferably such variants are at least 80%, such as at least 85%, such as at least 90, such as at least 95% identical to one of the alpha-1,3/4-fucosyltransferase in table 6.

TABLE 6
List of α-1,3/4-fucosyltransferase

| Protein | | | |
|---|---|---|---|
| Name | GenBank ID | Origin | HMO product |
| FucTIII | AY450598.1 | Helicobacter | LNFP-II, LNFP-V, |
| | | pylori strain | LNDFH-I, |
| | | DSM 6709 | LNDFH-II, |
| FutA | WP_000487428.1 | Helicobacter | DFL, LNFP-V, |
| | | pylori | LNDFH-II |
| | | ATCC 26695 | |

### Alpha-2,3-Sialyltransferase

An α-2,3-sialyltransferase refer to a glycosyltransferase that catalyzes the transfer of sialyl from a donor substrate for example, CMP-N-acetylneuraminic acid, to an acceptor molecule in an alpha-2,3-linkage. Preferably, an alpha-2,3-sialyltransferase used herein does not originate in the species of the genetically engineered cell i.e., the gene encoding the 2,3-sialyltransferase is of heterologous origin. Non-limiting examples α-2,3-sialyltransferase are given in table 7. α-2,3-sialyltransferase variants may also be useful, preferably such variants are at least 80%, such as at least 85%, such as at least 90, such as at least 95% identical to one of the α-2,3-sialyltransferase in table 7.

TABLE 7
List of α-2,3-sialyltransferase

| Protein Name | GenBank ID | Origin | HMO product |
|---|---|---|---|
| Avi | WP_115249238.1 | Avibacterium avium | 3′-SL |
| Ccol | WP_075498955.1 | Campylobacter coli | 3′-SL, FSL |
| Ccol2 | EAH6554614.1 | Campylobacter coli | 3′-SL, 6′SL, LST-a |
| Chepa | WP_066776435.1 | Campylobacter hepaticus | 3′-SL, 6′SL, LST-a |
| Cinf1 | WP_011272254.1 | Haemophilus influenzae | 3′-SL, 6′SL |
| Cjej1 | EBD1936710.1 | Campylobacter jejuni | 3′-SL, 6′SL, LST-a |
| Clari1 | EGK8106227.1 | Campylobacter lari | 3′-SL, LST-a |
| Cstl | AAF13495.1 | Campylobacter jejuni | 3′-SL, LST-a |
| Cstll | AAF31771.1 | Campylobacter jejuni | 3′-SL, 6′SL, LST-a |
| Csub1 | WP_039664428.1 | Campylobacter subantarcticus | 3′-SL |
| MhnNBse | WP_176810284.1 | Mannheimia (multispecies) | 3′-SL |
| Neigon | AAW89748.1 | Neisseria gonorrhoeae FA 1090 | 3′-SL |
| Nst | AAC44541.1 | Neisseria meningitidis MC58 | 3′-SL |
| PM70 | AAK03258.1 | Pasteurella multocida subsp. | 3′-SL |
| | | multocida str. Pm70 | |
| PmN | WP_005726268.1 | Pasteurella (multispecies) | 3′-SL |
| Pmult | WP_005753497.1 | Pasteurela multocida | 3′-SL |
| Poral | WP_101774487.1 | Pasteurella oralis | 3′-SL |
| Poral_2 | WP_101774701.1 | Pasteurella oralis | 3′-SL, 6′SL |

### Alpha-2, 6-Sialyltransferase

An alpha-2,6-sialyltransferase refer to a glycosyltransferase that catalyzes the transfer of sialyl from a donor substrate for example, CMP-N-acetylneuraminic acid, to an acceptor molecule in an alpha-2,6-linkage. Preferably, an alpha-2,6-sialyltransferase used herein does not originate in the species of the genetically engineered cell i.e., the gene encoding the 2,6-sialyltransferase is of heterologous origin. Non-limiting examples α-2,6-sialyltransferase are given in table 8. α-2,6-sialyltransferase variants may also be useful, preferably such variants are at least 80%, such as at least 85%, such as at least 90, such as at least 95% identical to one of the α-2,6-sialyltransferase in table 8.

TABLE 8
List of α-2,6-sialyltransferase

| Protein | | | HMO |
|---|---|---|---|
| Name | GenBank ID | Origin | product |
| HAC1268 | STP03859.1 | Helicobacter acinonychis | LST-c |
| | | str. Sheeba | |
| Pts6-119 | AB500947.1 | Photobacterium leiognathi | 6′SL, |
| | | JT-SHIZ-119 | LST-b |
| Pts6-224 | BAF92026.1 | Photobacterium sp | 6, SL, LST-b, |
| | | JT-ISH-224 | DSLNT |
| PiST6_145 | BAF91416.1 | Photobacterium leiognathi | 6′SL, |
| | | | LST-b |

### Glycosyl-Donor—Nucleotide-Activated Sugar Pathways

When carrying out the method of this present disclosure, preferably a glycosyltransferase mediated glycosylation reaction takes place in which an activated sugar nucleotide serves as glycosyl-donor. An activated sugar nucleotide generally has a phosphorylated glycosyl residue attached to a nucleoside. A specific glycosyl transferase enzyme accepts only a specific sugar nucleotide. Thus, preferably the following activated sugar nucleotides are involved in the glycosyl transfer: glucose-UDP-GlcNAc, UDP-galactose, UDP-glucose, UDP-N-acetylglucosamine, UDP-N-acetylgalactosamine (GlcNAc) and CMP-N-acetylneuraminic acid. The genetically modified cell according to the present disclosure can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid (CMP-Neu5Ac). In table 9 below are non-limiting examples of glycosyl-donors and the HMO products they can be used to produce, the list may not be exhaustive.

TABLE 9
glycosyl-donor HMO product list

| Glycosyl-donor | HMO product |
|---|---|
| UDP-GlcNAc | LNT, LNnT, LNFP-I, LNFP-II, LNFP-III, |
| | LNFP-V, LNFP-VI, LNDFH-I, LNDFH-II, LNDFH-III, |
| | LNH, LNnH, pLNH, pLNnH, F-pLNH-I, F-pLNH-II, |
| | F-pLNH-I, F-pLNnH-II, FLSTa, FLSTb, FLSTc, |
| | FLSTd, LSTa, LSTb, LSTc, LSTd, DSLNT, |
| | SLNH, SLNH-II |
| UDP-Gal | LNT, LNnT, LNFP-I, LNFP-II, LNFP-III, LNFP-V, |
| | LNFP-VI, LNDFH-I, LNDFH-II, LNDFH-III, LNH, |
| | LNnH, pLNH, pLNnH, F-pLNH-I, F-pLNH-II, |
| | F-pLNH-I, F-pLNnH-II, FLSTa, FLSTb, FLSTc, |
| | FLSTd, LSTa, LSTb, LSTc, LSTd, DSLNT, SLNH-I, |
| | SLNH-II |
| GDP-fucose | 2′FL, 3FL, DFL, LNFP-I, LNFP-II, LNFP-III, |
| | LNFP-V, LNFP-VI, LNDFH-I, LNDFH-II, LNDFH-III, |
| | F-LNH, F-LNnH, F-pLNH-I, F-pLNH-II, F-pLNH-I, F- |
| | pLNnH-II, FSL, FLSTa, FLSTb, FLSTc, FLSTd |
| CMP-Neu5Ac | 3′SL, 6′SL, FSL, FLSTa, FLSTb, FLSTc, |
| | FLSTd, LSTa, LSTb, LSTc, LSTd, DSLNT, SLNH-I, |
| | SLNH-II |

In one embodiment of the method, the genetically modified cell is capable of producing one or more activated sugar nucleotides mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, sucrose, fructose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009)).

The enzymes involved in the de novo biosynthetic pathway of an activated sugar nucleotide can be naturally present in the cell or introduced into the cell by means of gene technology or recombinant DNA techniques, all of them are parts of the general knowledge of the skilled person.

In another embodiment, the genetically modified cell can utilize salvaged monosaccharides for sugar nucleotide. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the host cell.

### Colanic Acid Gene Cluster

For the production of fucosylated HMO's the colanic acid gene cluster is important to ensure presence of sufficient GDP-fucose. In Escherichia coli GDP-fucose is an intermediate in the production of the extracellular polysaccharide colanic acid, a major oligosaccharide of the bacterial cell wall. In the context of the present disclosure the colanic acid gene cluster encodes the enzymes involved in the de novo synthesis of GDP-fucose, whereas one or several of the genes downstream of GDP-L-fucose, such as wcaJ, can be deleted to prevent conversion of GDP-fucose to colanic acid. The colanic acid gene cluster responsible for the formation of GDP-fucose comprises or consists of the genes: gmd which encodes the protein GDP-mannose-4,6-dehydratase, wcaG (fcl) which encodes the protein GDP-L-fucose synthase, wcaH which encodes the protein GDP-mannose mannosyl hydrolase, wcal which encodes the colanic acid biosynthesis glycosyltransferase, manB which encodes the protein phosphomannomutase and manC which encodes the protein mannose-1-phosphate guanylyltransferase.

In one or more exemplary embodiment(s), the colanic acid gene cluster responsible for the formation of GDP-fucose may be expressed from its native genomic locus. The expression may be actively modulated to increase GDP-fucose formation. The expression can be modulated by swapping the native promoter with a promoter of interest, and/or increasing the copy number of the colanic acid genes coding said protein(s) by expressing the gene cluster from another genomic locus than the native, or episomally expressing the colanic acid gene cluster or specific genes thereof.

In relation to the present disclosure, the term “native genomic locus”, in relation to the colanic acid gene cluster, relates to the original and natural position of the gene cluster in the genome of the genetically engineered cell.

The de novo GDP-fucose pathway genes responsible for the formation of GDP-fucose comprises or consists of the following genes:
i) manA which encodes the protein mannose-6 phosphate isomerase (EC 5.3.1.8, UniProt accession nr. P00946), which facilitates the interconversion of fructose 6-phosphate (F6P) and mannose-6-phosphate;
ii) manB which encodes the protein phosphomannomutase (EC 5.4.2.8, UniProt accession nr P24175), which is involved in the biosynthesis of GDP-mannose by catalyzing conversion mannose-6-phosphate into mannose-1-phosphate;
iii) manC which encodes the protein mannose-1-phosphate guanylyltransferase guanylyltransferase (EC:2.7.7.13, UniProt accession nr P24174), which is involved in the biosynthesis of GDP-mannose through synthesis of GDP-mannose from GTP and α-D-mannose-1-phosphate;
iv) gmd which encodes the protein GDP-mannose-4,6-dehydratase (UniProt accession nr POAC88), which catalyzes the conversion of GDP-mannose to GDP-4-dehydro-6-deoxy-D-mannose;
v) wcaG (fcl) which encodes the protein GDP-L-fucose synthase (EC 1.1.1.271, UniProt accession nr P32055) which catalyses the two-step NADP-dependent conversion of GDP-4-dehydro-6-deoxy-D-mannose to GDP-fucose.

Accordingly, it is preferred that the genetically engineered cell, when producing one or more fucosylated heterologous products, overexpresses either the entire colonic acid gene cluster and/or one or more genes of the de novo GDP-fucose pathway selected from the group consisting of manA, manB, manC, gmd and wcaG.

### Sialic Acid Sugar Nucleotide Synthesis Pathway

If the genetically modified cell is to produce a sialylated HMO the genetically modified cell comprises a sialic acid sugar nucleotide synthesis capability, i.e., the genetically modified cell comprises a biosynthetic pathway for making a sialate sugar nucleotide, such as CMP-N-acetylneuraminic acid as glycosyl-donor for the sialyltransferases. E.g., the genetically modified cell comprises a sialic acid synthetic capability through provision of an exogenous UDP-GlcNAc 2-epimerase (e.g., neuC of Campylobacter jejuni (GenBank AAK91727.1) or equivalent (e.g., (GenBank CAR04561.1), a Neu5Ac synthase (e.g., neuB of C. jejuni (GenBank AAK91726.1) or equivalent, (e.g., Flavobacterium limnosediminis sialic acid synthase, GenBank WP_023580510.1), and/or a CMP-Neu5Ac synthetase (e.g., neuA of C. jejuni (GenBank AAK91728.1) or equivalent, (e.g., Vibrio brasiliensis CMP-sialic acid synthase, GenBank WP_006881452.1).

Furthermore, the genetically modified cell preferably has a deficient sialic acid catabolic pathway. By “sialic acid catabolic pathway” is meant a sequence of reactions, usually controlled, and catalysed by enzymes, which results in the degradation of sialic acid. An exemplary sialic acid catabolic pathway described hereafter is the E. coli pathway. In this pathway, sialic acid (Neu5Ac; N-acetylneuraminic acid) is degraded by the enzymes NanA (N-acetylneuraminic acid lyase) and NanK (N-acetylmannosamine kinase) and NanE (N-acetylmannosamine-6-phosphate epimerase), all encoded from the nanATEK-yhcH operon, and repressed by NanR (http://ecocyc.org/ECOLI). A deficient sialic acid catabolic pathway is rendered in the E. coli host by introducing a mutation in the endogenous nanA (N-acetylneuraminate lyase) (e.g., GenBank Accession Number D00067.1(GL216588)) and/or nanK (N-acetylmannosamine kinase) genes (e.g., GenBank Accession Number (amino acid) BAE77265.1 (GL85676015)), and/or nanE (N-acetylmannosamine-6-phosphate epimerase, GI: 947745), incorporated herein by reference). Optionally, the nanT (N-acetylneuraminate transporter) gene is also inactivated or mutated. Other intermediates of sialic acid metabolism include: (ManNAc-6-P) N-acetylmannosamine-6-phosphate; (GlcNAc-6-P) N-acetylglucosamine-6-phosphate; (GlcN-6-P) Glucosamine-6-phosphate, and (Fruc-6-P) Fructose-6-phosphate. In some preferred embodiments, nanA is mutated. In other preferred embodiments, nanA and nanK are mutated, while nanE remains functional. In another preferred embodiment, nanA and nanE are mutated, while nanK has not been mutated, inactivated or deleted. A mutation is one or more changes in the nucleic acid sequence coding the gene product of nanA, nanK, nanE, and/or nanT. E.g., the mutation may be 1, 2, up to 5, up to 10, up to 25, up to 50 or up to 100 changes in the nucleic acid sequence. E.g., the nanA, nanK, nanE, and/or nanT genes are mutated by a null mutation. Null mutations as described herein encompass amino acid substitutions, additions, deletions, or insertions, which either cause a loss of function of the enzyme (i.e., reduced or no activity) or loss of the enzyme (i.e., no gene product). By “deleted” is meant that the coding region is removed completely or in part such that no (functional) gene product is produced. By inactivated is meant that the coding sequence has been altered such that the resulting gene product is functionally inactive or encodes for a gene product with less than 100%, e.g., 90%, 80%, 70%, 60%, 50%, 40%, 30% or 20% of the activity of the native, naturally occurring, endogenous gene product. Thus, in the present disclosure, nanA, nanK, nanE, and/or nanT genes are preferably inactivated.

### Hmo Transporter Proteins

The term “HMO transporter” means a biological molecule, e.g. protein, that facilitates transport/export an HMO synthesized by the host cell through a cellular membrane, e.g. into the cell medium, or transport/import of an HMO from the cell medium into the cell cytosol.

The oligosaccharide product, such as the HMO produced by the cell, can be accumulated both in the intra- and the extracellular matrix. The product can be transported to the supernatant in a passive way, i.e., it diffuses outside across the cell membrane. The more complex HMO products may remain in the cell, which is likely to eventually impair cellular growth, thereby affecting the possible total yield of the product from a single fermentation. The HMO transport can be facilitated by major facilitator superfamily transporter proteins (MFS transporter).

The term “MFS transporter” in the present context means a protein that facilitates transport of an oligosaccharide, preferably, an HMO, through or across the cell membrane, preferably of an HMO/oligosaccharide synthesized by the genetically engineered cell as described herein from the cell cytosol to the cell medium. Additionally, or alternatively, the MFS transporter may also facilitate efflux of molecules that are not considered HMO or oligosaccharides, such as lactose, glucose, cell metabolites and/or toxins. The specificity towards the sugar moiety of the product to be secreted can be altered by mutation by means of known recombinant DNA techniques.

Thus, in one or more exemplary embodiments, the genetically engineered cell according to the method described herein further comprises a gene product that acts as a major facilitator superfamily transporter. The gene product that acts as a major facilitator superfamily transporter may be encoded by a recombinant nucleic acid sequence that is expressed in the genetically engineered cell. The recombinant nucleic acid sequence encoding a major facilitator superfamily transporter, may be integrated into the genome of the genetically engineered cell, or expressed using a plasmid.

In one or more exemplary embodiment(s), the MFS transporter is selected from the group consisting of Bad, Nec, YberC, Fred, Vag and Marc.

The genetically engineered cell of the present disclosure thus in one or more exemplary embodiment(s) expresses a heterologous MFS transporter protein selected from the group consisting of Vag (GenBank accession ID WP_048785139.1), Nec (GenBank accession ID WP_092672081.1), Fred (GenBank accession ID WP_087817556.1), Marc (GenBank accession WP_060448169.1), YberC (GenBank accession ID EEQ08298.1), Bad (GenBank accession ID WP_017489914.1) and a functional homologue of any one of Vag, Nec, Fred, Marc, YberC or Bad having an amino acid sequence which is 70% identical to the amino acid sequence of any one of Vag, Nec, Fred, Marc, YberC or Bad.

In some embodiment the genetically modified cell of the present disclosure comprises a nucleic acid sequence encoding a putative MFS (major facilitator superfamily) transporter protein, originating from the bacterium Rosenbergiella nectarea. More specifically, the present disclosure relates to a genetically modified cell optimized to produce an HMO, comprising a recombinant nucleic acid encoding MFS transporter protein having at least 80%, such as at least 85%, such as at least 90% such as at least 95% or 100% sequence identity to the amino acid sequence of the amino acid sequence having GenBank accession ID WP_092672081.1 (nec). Additionally, the MFS transporter protein with the GenBank accession ID WP_092672081.1 is further described in WO2021/148615.

Nec is expected to facilitate an increase in the efflux of the produced sialylated HMOs, e.g., 3′SL in the genetically engineered cells of the present disclosure.

In some embodiments, the genetically modified cell of the present disclosure comprises a nucleic acid sequence encoding a putative MFS (major facilitator superfamily) transporter protein, originating from the bacterium Yersinia frederiksenii and/or the bacterium Yersinia bercovieri. More specifically, the present disclosure relates to a genetically modified cell optimized to produce an HMO, comprising a recombinant nucleic acid encoding a MFS transporter protein having at least 80%, such as at least 85%, such as at least 90% such as at least 95% or 100% sequence identity to the amino acid sequence of the amino acid sequence having the GenBank accession ID WP_087817556.1 (fred) or GenBank accession EEQ08298 (YberC). The MFS transporter protein with the GenBank accession ID WP_087817556.1 is further described in WO2021/148620 and the MFS transporter protein with the GenBank accession ID EEQ08298 is further described in WO2021/148610.

In some embodiments, the genetically modified cell of the present disclosure comprises a nucleic acid sequence encoding a putative MFS (major facilitator superfamily) transporter protein, originating from the bacterium Serratia marcescens. More specifically, the present disclosure relates to a genetically modified cell optimized to produce an HMO, comprising a recombinant nucleic acid encoding MFS transporter protein having at least 80%, such as at least 85%, such as at least 90% such as at least 95% or 100% sequence identity to the amino acid sequence of the amino acid sequence having the GenBank accession WP_060448169.1 (marc). The MFS transporter protein with the GenBank accession ID WP_087817556.1 is further described in WO2021/148614.

In some embodiments, the genetically modified cell of the present disclosure comprises a nucleic acid sequence encoding a putative MFS (major facilitator superfamily) transporter protein, originating from the bacterium Pantoea vagans. More specifically, the present disclosure relates to a genetically modified cell optimized to produce an HMO, comprising a recombinant nucleic acid encoding MFS transporter protein having at least 80%, such as at least 85%, such as at least 90% such as at least 95% or 100% sequence identity to the amino acid sequence of the amino acid sequence having the GenBank accession WP_048785139.1 (vag). The MFS transporter protein with the GenBank accession ID WP_048785139.1 is further described in WO2021/148611.

### The Genetically Modified Cell

In the present context, the terms “a genetically modified cell” and “a genetically engineered cell” are used interchangeably. As used herein “a genetically modified cell” is a host cell whose genetic material has been altered by human intervention using a genetic engineering technique, such a technique is e.g., but not limited to transformation or transfection e.g., with a heterologous polynucleotide sequence, Crisper/Cas editing and/or random mutagenesis. In one embodiment the genetically engineered cell has been transformed or transfected with a recombinant nucleic acid sequence.

In one aspect of the present disclosure the genetically modified cell is capable of producing one or more Human Milk Oligosaccharides (HMOs), wherein said cell comprises the following modifications: a) overexpression of citrate synthase (gltA), b) one or more heterologous nucleic acids encoding one or more glycosyltransferases, and c) at least one biosynthetic pathway for making an activated sugar nucleotide capable of serving as glycosyl-donor for the glycosyl transferase(s) of b).

In a further aspect of the present disclosure the genetically modified cell is capable of producing one or more Human Milk Oligosaccharides (HMOs), wherein said cell comprises the following modifications: a) overexpression of citrate synthase (gltA), and b) one or more heterologous nucleic acids encoding one or more glycosyltransferases, and c) at least one biosynthetic pathway for making an activated sugar nucleotide capable of serving as glycosyl-donor for the glycosyl transferase(s) of b) and d) at least one of the following modifications: i) overexpression of phosphoenolpyruvate carboxylase (ppc), and/or ii) decreased or total loss of function of the isocitrate lyase regulator (iclR).

In addition to the genetic modification in the gltA gene and ppc gene and/or iclR gene of the present disclosure further genetic modifications can e.g., be selected from glycosyltransferases, and/or metabolic pathway engineering and inclusion of MFS transporters as described in the above sections, which the skilled person will know how to combine into a genetically modified cell capable of producing one or more HMOs of interest.

The genetically modified cell comprising more than one glycosyltransferase activity described herein will generally produce a mixture of two or more HMOs, whereas cells with a single glycosyltransferase activity generally produce one or maximum two HMOs.

In one embodiment of the present disclosure, the genetically modified cell comprises a recombinant nucleic acid sequence encoding a citrate synthase comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1.

In one embodiment of the present disclosure the recombinant nucleic acid sequence encoding a citrate synthase citrate comprise the native gltA gene in its native locus under control of a promoter that is stronger than the native promoter.

In another embodiment of the present disclosure the recombinant nucleic acid encoding a citrate synthase is inserted into the genome of the cell as an additional copy to the native gltA gene, either in the same locus or in a different locus of the genome or as an episomal copy e.g. on a plasmid. If the host cell does not contain a native gltA gene, a heterologous nucleic acids encoding a citrate synthase is inserted into a suitable locus of the genome or as an episomal copy e.g. on a plasmid. In a preferred embodiment the promoter controlling the expression of the citrate synthase is selected from table 10.

In a further embodiment of the present disclosure, the genetically modified cell comprises a recombinant nucleic acid sequence encoding phosphoenolpyruvate carboxylase comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2.

In one embodiment of the present disclosure the recombinant nucleic acid sequence encoding a phosphoenolpyruvate carboxylase comprise the native ppc gene in its native locus under control of a promoter that is stronger than the native promoter.

In another embodiment of the present disclosure the recombinant nucleic acid encoding a phosphoenolpyruvate carboxylase is inserted into the genome of the cell as an additional copy to the native ppc gene, either in the same locus or in a different locus of the genome or as an episomal copy e.g. on a plasmid. If the host cell does not contain a native ppc gene, a heterologous nucleic acids encoding a citrate synthase is inserted into a suitable locus of the genome or as an episomal copy e.g. on a plasmid. In a preferred embodiment the promoter controlling the expression of the citrate synthase is selected from table 10.

In one embodiment of the present disclosure, the genetically modified cell comprises a) a recombinant nucleic acid sequence encoding a citrate synthase comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1 and b) a recombinant nucleic acid sequence encoding phosphoenolpyruvate carboxylase comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2, in addition to the modifications needed to produce one or more HMOs of interest.

In one embodiment of the present disclosure, the genetically modified cell comprises a) a recombinant nucleic acid sequence encoding a citrate synthase comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1 and b) a deletion or loss of function of the iclR gene, in addition to the modifications needed to produce one or more HMOs of interest.

In one embodiment of the present disclosure, the genetically modified cell comprises a) a recombinant nucleic acid sequence encoding a citrate synthase comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1 and b) a recombinant nucleic acid sequence encoding phosphoenolpyruvate carboxylase comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2, and c) a deletion or loss of function of the iclR gene, in addition to the modifications needed to produce one or more HMOs of interest.

In one embodiment of the present disclosure, the genetically modified cell comprises a) a recombinant nucleic acid sequence encoding a phosphoenolpyruvate carboxylase comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2 and b) a deletion or loss of function of the iclR gene, in addition to the modifications needed to produce one or more HMOs of interest.

The genetically engineered cell is preferably a microbial cell, such as a prokaryotic cell or eukaryotic cell. Appropriate microbial cells that may function as a host cell include bacterial cells, archaebacterial cells and algae cells and fungal cells.

In one preferred embodiment, the genetically engineered cell is a bacterial cell which in its native state express a protein of the isocitrate lyase regulator family.

### Host Cells

The genetically engineered cell may be any cell useful for HMO production including mammalian cell lines. Preferably, the host cell is a unicellular microorganism of eucaryotic or prokaryotic origin. Appropriate microbial cells that may function as a host cell include yeast cells, bacterial cells, archaebacterial cells, algae cells, and fungal cells.

The genetically engineered cell (host cell) may be e.g., a bacterial or yeast cell. In one preferred embodiment, the genetically engineered cell is a bacterial cell.

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) could be Escherichia coli, Erwinia herbicola (Pantoea agglomerans), Citrobacter freundii, Campylobacter sp, Corynebacterium sp., Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods described herein, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles, Streptomyces lividans, and Proprionibacterium freudenreichii are also suitable bacterial species. Also included are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of a HMO product could be yeast cells, such as Komagataella phaffii, Kluyveromyces lactis, Yarrowia lipolytica, Pichia pastoris, and Saccharomyces cerevisiae or filamentous fungi such as Aspergillus sp, Fusarium sp or Trichoderma sp, exemplary species are A. niger, A. nidulans, A. oryzae, F. solani, F. graminearum and T. reesei.

In one or more exemplary embodiments, the genetically engineered cell is selected from the group consisting of Yarrowia lipolytica, Pichia pastoris, and Saccharomyces cerevisiae.

In one or more exemplary embodiments, the genetically engineered cell is Pichia pastoris.

In one or more exemplary embodiments, the genetically engineered cell is selected from the group consisting of Escherichia sp., Bacillus sp., Lactobacillus sp and Corynebacterium sp. Campylobacter sp.

In one or more exemplary embodiments, the genetically engineered cell is selected from the group consisting of E. coli, C. glutamicum, L. lactis, B. subtilis, S. lividans.

In one or more exemplary embodiments, the genetically engineered cell is Bacillus subtilis.

In one or more exemplary embodiments, the genetically engineered cell is Corynebacterium glutamicum.

In one or more exemplary embodiments, the genetically engineered cell is Escherichia coli.

In one or more exemplary embodiments, the genetically engineered cell is derived from the E. coli K-12 strain or E. coli DE3.

### A Recombinant Nucleic Acid Sequence

In the present context, the term “recombinant nucleic acid sequence”, “recombinant gene/nucleic acid/nucleotide sequence/DNA encoding” or “coding nucleic acid sequence” is used interchangeably and intended to mean an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e., a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The recombinant nucleic acid sequence may be a coding DNA sequence e.g., a gene, or non-coding DNA sequence e.g., a regulatory DNA, such as a promoter sequence or other non-coding regulatory sequences.

The recombinant nucleic acid sequence may in addition be heterologous. As used herein “heterologous” refers to a polypeptide, amino acid sequence, nucleic acid sequence or nucleotide sequence that is foreign to a cell or organism, i.e., to a polypeptide, amino acid sequence, nucleic acid molecule or nucleotide sequence that does not naturally occurs in said cell or organism.

The present disclosure also relates to a nucleic acid construct comprising a coding nucleic sequence, i.e. recombinant DNA sequence of a gene of interest, e.g., a citrate synthase and/or a phosphoenolpyruvate carboxylase, and a non-coding regulatory DNA sequence, e.g., a promoter DNA sequence, e.g., a recombinant promoter sequence derived from the promoter sequence of the lac operon or the glp operon, or a promoter sequence derived from another genomic promoter DNA sequence, or a synthetic promoter sequence, wherein the coding and promoter sequences are operably linked.

The term “operably linked” refers to a functional relationship between two or more nucleic acid (e.g., DNA) segments. It refers to the functional relationship of a transcriptional regulatory sequence to a transcribed sequence. E.g., a promoter sequence is operably linked to a coding sequence if it stimulates or modulates the transcription of the coding sequence in an appropriate host cell or other expression system.

Generally, promoter sequences that are operably linked to a transcribed sequence are physically contiguous to the transcribed sequence, i.e., they are cis-acting.

In one exemplified embodiment, the nucleic acid construct of the present disclosure may be a part of the vector DNA, in another embodiment, the construct it is an expression cassette/cartridge that is integrated in the genome of a host cell.

Accordingly, the term “nucleic acid construct” means an artificially constructed segment of nucleic acids, in particular a DNA segment, which is intended to be inserted into a target cell, e.g., a bacterial cell, to modify expression of a gene of the genome or expression of a gene/coding DNA sequence which may be included in the construct. Thus, in embodiments, the present disclosure relates to a nucleic acid construct comprising a recombinant nucleic acid sequence encoding a citrate synthase and/or a phosphoenolpyruvate carboxylase, wherein said recombinant nucleic acid sequence is selected from the group consisting of nucleic acid sequences encoding a protein of SEQ ID NO: 1 and/or SEQ ID NO: 2, or functional variants thereof.

One embodiment of the present disclosure is a nucleic acid construct comprising a recombinant nucleic acid sequence encoding a citrate synthase, wherein said recombinant nucleic acid sequence comprise or consist of the nucleic acid sequences of SEQ ID NO: 3 or an nucleic acid sequence with at least 80%, such as at least 85%, such as at least 90%, such as at least 95%, or such as at least 99% identity to SEQ ID NO: 3. Preferably, the citrate synthase encoding sequence is under the control of a promoter sequence selected from promotor sequences with a nucleic acid sequence as identified in Table 10.

Another embodiment of the present disclosure is a nucleic acid construct comprising a recombinant nucleic acid sequence encoding a phosphoenolpyruvate carboxylase, wherein said recombinant nucleic acid sequence comprise or consist of the nucleic acid sequences of SEQ ID NO: 4 or an nucleic acid sequence with at least 80%, such as at least 85%, such as at least 90%, such as at least 95%, or such as at least 99% identity to SEQ ID NO: 4. Preferably, the phosphoenolpyruvate carboxylase encoding sequence is under the control of a promoter sequence selected from promotor sequences with a nucleic acid sequence as identified in Table 10.

TABLE 10
Selected promoter sequences

| | % Activity | | | |
|---|---|---|---|---|
| | relative | | | Seq ID |
| Promoter name | to PglpF* | Strength | Reference | in appl. |
| PmglB_70UTR_SD8 | 291% | high | WO2020255054 | 5 |
| PmglB_70UTR_SD10 | 233-281% | high | WO2020255054 | 6 |
| PmglB_54UTR | 197% | high | WO2020255054 | 7 |
| Plac_70UTR | 182-220% | high | WO2019123324 | 8 |
| PmglB_70UTR_SD9 | 180-226% | high | WO2020255054 | 9 |
| PmglB_70UTR_SD4 | 153%-353% | high | WO2020255054 | 10 |
| PmglB_70UTR_SD5 | 146-152% | high | WO2020255054 | 11 |
| PglpF_SD4 | 140-161% | high | WO2019123324 | 12 |
| PmglB_70UTR_SD7 | 127-173% | high | WO2019123324 | 13 |
| PmglB_70UTR | 124-234% | high | WO2020255054 | 14 |
| PglpA_70UTR | 102-179% | high | WO2019123324 | 15 |
| PglpT_70UTR | 102-240% | high | WO2019123324 | 16 |
| PglpF | 100% | high | WO2019123324 | 17 |
| PglpF_SD10 | 88-96% | high | WO2019123324 | 18 |
| PglpF_SD5 | 82-91% | high | WO2019123324 | 19 |
| PglpF_SD8 | 81-82% | high | WO2019123324 | 20 |
| Pcon3_70UTR | 79-99% | high | | 29 |
| PmglB_16UTR | 78-171% | high | WO2019123324 | 21 |
| PglpF_SD9 | 73-93% | middle | WO2019123324 | 22 |
| PglpF_SD7 | 47-57% | middle | WO2019123324 | 23 |
| PglpF_SD6 | 46-47% | middle | WO2019123324 | 24 |
| PglpA_16UTR | 38-64% | middle | WO2019123324 | 25 |
| Plac_wt* | 15-28% | low | WO2019123324 | 26 |
| PglpF_SD3 | 9% | low | WO2019123324 | 27 |
| PglpF_SD1 | 5% | low | WO2019123324 | 28 |

*The promoter activity is assessed in the LacZ assay described below with the PglpF promoter run as positive reference in the same assay. To compare across assays the activity is calculated relative to the PglpF promoter, a range indicates results from multiple assays.

The promoter may be of heterologous origin, native to the genetically modified cell or it may be a recombinant promoter, combining heterologous and/or native elements.

One way to increase the production of a product may be to regulate the production of the desired enzyme activity used to produce the product, such as the glycosyltransferases or enzymes involved in the biosynthetic pathway of the glycosyl donor.

Increasing the promoter strength driving the expression of the desired enzyme may be one way of doing this. The strength of a promoter can be assed using a lacZ enzyme assay where β-galactosidase activity is assayed as described previously (see e.g. Miller J. H. Experiments in molecular genetics, Cold spring Harbor Laboratory Press, NY, 1972). Briefly the cells are diluted in Z-buffer and permeabilized with sodium dodecyl sulfate (0.1%) and chloroform. The LacZ assay is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU.

In embodiments the expression of said nucleic acid sequences of the present disclosure is under control of a PglpF (SEQ ID NO: 17) or Plac (SEQ ID NO: 26) or PmglB_UTR70 (SEQ ID NO: 14) or PglpA_70UTR (SEQ ID NO: 15) or PglpT_70UTR (SEQ ID NO: 16) or Pcon3_70UTR (SEQ ID NO: 29) or variants of these promoters as identified in Table 10, in particular PglpF variants of SEQ ID NO: 12, 18, 19, 20, 22, 23 or 24 or Plac variant of SEQ ID NO: 8 or PmglB_70UTR variants of SEQ ID NO: 5, 6, 7, 9, 10, 11, 13 or 21. Further suitable variants of PglpF, PglpA_70UTR, PglpT_70UTR and Pmg|B_70UTR promoter sequences are described in or WO2019/123324 and WO2020/255054 respectively (hereby incorporated by reference).

Integration of the nucleic acid construct of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February; 2(2):137-49); methods for genomic integration of nucleic acid sequences in which recombination is mediated by the Red recombinase function of the phage A or the RecE/RecT recombinase function of the Rac prophage (Murphy, J Bacteriol. (1998); 180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. (2005), 12(3):349-56; Vetcher et al., Appl Environ Microbiol. (2005); 71(4):1829-35); or positive clones, i.e., clones that carry the expression cassette, can be selected e.g., by means of a marker gene, or loss or gain of gene function.

In one or more exemplary embodiments, the present disclosure relates to a nucleic acid construct comprising one or more recombinant nucleic acid sequences as illustrated in SEQ ID NO: 3 or 4 [nucleic acid encoding gltA or ppc, respectively] and a promoter sequence selected from table 10 at the 5′ end of SEQ ID NO: 3 or 4.

In a specific embodiment SEQ ID NO: 3 and/or 4 is under control of the Pcon3_70UTR promoter (SEQ ID NO: 29).

In a specific embodiment SEQ ID NO: 3 and/or 4 is under control of the PglpF promoter (SEQ ID NO: 17).

In particular, the present disclosure relates to one or more of a recombinant nucleic acid sequence and/or to a functional homologue thereof having a sequence which is at least 70% identical to SEQ ID NO: 3 or 4 [nucleic acids encoding gltA or ppc, respectively], such as at least 75% identical, at least 80% identical, at least 85% identical, at least 90% identical, at least, at least 95% identical, at least 98% identical, or 100% identical.

### Sequence Identity

The term “sequence identity” as used herein describes the relatedness between two amino acid sequences or between two nucleotide sequences, i.e., a candidate sequence (e.g., a sequence of the disclosure) and a reference sequence (such as a prior art sequence) based on their pairwise alignment. For purposes of the present disclosure, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the -nobrief option) is used as the percent identity and is calculated as follows: (Identical Residues×100)/(Length of Alignment−Total Number of Gaps in Alignment).

For purposes of the present disclosure, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides×100)/(Length of Alignment−Total Number of Gaps in Alignment).

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality/activity of at least 50%, such as at least 60%, 70%, 80%, 90% or 100% compared to the functionality of the protein/nucleic acid sequence.

A functional homologue with a decrease in remaining functionality/activity will be considered as a variant with decreased function. Functional homologues or variants with decreased function can for example be obtained by introducing one or more missense mutations in the encoding gene that results in a change in the amino acid sequence that reduces its functionality of the expressed polypeptide. Variants, where the encoded polypeptide maintain less than 5% functionality/activity are considered as having total loss of function. Introducing of a nonsense mutation resulting in a premature stop codon rendering the transcribed polypeptide dysfunctional can be used to generate host cells with total loss of function. Total loss of function can also be obtained by deleting the gene encoding the polypeptide of interest.

In one embodiment of the present disclosure the host cell has decreased or total loss of function of the isocitrate lyase regulator (IclR). In a preferred embodiment the iclR gene has been deleted from the host cell.

A functional homologue of any one of the disclosed amino acid or nucleic acid sequences can also have a higher functionality. A functional homologue of any one of the proteins shown in table 1, 2, 3, 4, 5, 6, 7, 8 and 9 or a recombinant nucleic acid encoding any one of the proteins in these tables and should ideally be able to participate in the production of one or more HMOs. A functional homologue SEQ ID NO: 1 or SEQ ID NO: 2 with higher functionality is also encompassed by this invention since higher functionally may potentially achieve the same effect as the overexpression described herein.

### Use of a Genetically Modified Cell

The disclosure also relates to any commercial use of the genetically modified cell(s) or the nucleic acid construct(s) disclosed herein, such as, but not limited to, in a method for producing one or more human milk oligosaccharide (HMO).

In an embodiment of the present disclosure, the genetically modified cell and/or the nucleic acid construct according to the present disclosure is used in the manufacturing of HMOs. Preferably, the genetically modified cell is used in large scale manufacturing of one or more HMOs. In one embodiment of the present disclosure a large-scale fermentation reaches a final fermentation volume above 1,000 L, such as above 10,000 L, such as above 50,000 L, preferably above 100,000 L.

Specifically, the use of the genetically modified cells of the present disclosure allows for fermentations where significantly less acetate or ethanol is produced during the fermentation, in particular during the non-carbon limited growth phase of the fermentation (e.g the batch phase in a fed-batch fermentation), as compared to the same HMO producing cell that does not have a) overexpression of citrate synthase (gltA), or b) overexpression of citrate synthase (gltA) and phosphoenolpyruvate carboxylase (ppc), or c) overexpression of citrate synthase (gltA) and has decreased or total loss of function of the isocitrate lyase regulator (iclR) or d) overexpression of citrate synthase (gltA) and phosphoenolpyruvate carboxylase (ppc) and has decreased or total loss of function of the isocitrate lyase regulator (iclR).

Preferably, the acetate level is reduced by at least 30%, such as at least 40%, such as by at least 50%, such as by at least 60% such as by at least 70% as compared to a cell not containing the modification(s) of the present disclosure. If the fermentation is a batch fermentation the acetate level is measured when the carbon source (sugar) has been depleted. If the fermentation is a fed-batch or continuous fed-batch fermentation the acetate level is ideally measured when the carbon source is depleted, which is normally around the time when the feeding is initiated. Carbon source depletion in the fermentation can be determined by either direct measurements (HPLC, sticks etc) or by indications from on-line fermentation data such as increase in Dissolved Oxygen (DO) or decrease in rpm, or both, typically in combination with increase in pH. Alternatively, a drop in CER (CO2 evolution rate) or OUR (oxygen uptake rate) can be used as measurements for carbon source depletion. Preferably, the acetate level is measured according to the Stress test described in the Methods section.

For host cells where overflow metabolism leads to ethanol formation instead of or in addition to acetate formation, which is typical for yeast cells, the genetically modified cell of the present disclosure produces less ethanol (and less acetate) as compared to a cell not containing the modification(s) of the present disclosure.

In a further embodiment of the present disclosure the genetically modified cells of the present disclosure are used to increase robustness of the cells in large-scale fermentations, in particular when subjected to high gradients of the carbon source used to feed the cells. Ideally the formation of the HMO product is not significantly affected when the genetically modified cell of the present disclosure encounters a high carbon source gradient or a large pulse of carbon source, such as glucose and/or sucrose added to the fermentation.

In terms of the present disclosure carbon source gradients are localized areas in the fermenter where the concentration of the carbon source used to feed the cells are higher than the average concentration throughout the fermenter. Carbon source gradients are ubiquitous in large-scale fermentations in particular in the area around the feed inlet, but they may also occur in other areas of the fermenter due to insufficient mixing. Specifically, a carbon source gradient may be a localized area within the fermenter where the molar concentration of the carbon source is higher than the Ks of the bacterium's sugar uptake system, which will roughly translate to the onset of overflow metabolism. Preferably, a carbon source gradient is more than 2 times, such as 5 times, such as 10 times the Ks, which is where the acetate accumulation starts to become severe for the cells.

In the context of the present disclosure Ks refers to the substrate affinity, i.e. the substrate concentration at which the cells grow at half maximum specific growth rate in the Monod model which describes the relationship between growth rate and substrate concentration in a microbial population growing with a single limiting carbon source. E. coli growth behavior using the Monod model is for example described in Wick et al 2002, Microbiology 148, 2889-2902 and Senn et al 1994 Biochimica et Biophysica Acta 1201, 424-436.

In a preferred embodiment the modified organism of the present disclosure will produce less acetate or ethanol than the non-modified cell when exposed to such carbon source gradients. The amount of acetate or ethanol formed when a cell is exposed to a carbon source gradient will also be dependent on the time the cell spends in the gradient. Consequently, in addition to the concentration of the carbon source in the gradient the time the cell spends within such a gradient may also be taken into account when assessing the reduction of the acetate or ethanol formation. Preferably, the cell spends at least 10 sec, such as at least 15 sec, such as at least 20 sec, such as at least 30 sec in a carbon source gradient when accessing the level of acetate or ethanol reduction achieved by the modifications made to the cell. The time spent in a carbon source gradient depends on several factors for example the speed of the agitation in the bioreactor, where the carbon source inlet is placed in the reactor, the amount and concentration of carbon source feed to the reactor. In the context of the present disclosure carbon source gradients can be mimicked in smaller scale by applying a glucose pulse as described in the Stress test in the Methods section.

### A Method for Producing Human Milk Oligosaccharides (HMOs)

The present disclosure also relates to a method for producing a human milk oligosaccharide (HMO), said method comprises culturing a genetically modified cell according to the present disclosure in a suitable culture medium.

The method comprising culturing a genetically modified cell that produces a HMO and further comprises culturing said genetically engineered cell in in the presence of an energy source (carbon source) selected from the group consisting of glucose, sucrose, fructose, xylose and glycerol. Preferably, the carbon source is one that results in overflow metabolism when present in excess amounts during fermentation. Preferably, the carbon source is glucose or sucrose, including hydrolyzed sucrose.

In preferred embodiments the cultivation is a fed batch fermentation or a continuous fed-batch (feed and bleed fermentation), where the carbon source is continuously feed to the fermentation broth during the fermentation.

In one aspect, the method of the present disclosure further comprises providing an acceptor saccharide as substrate for the HMO formation, the acceptor saccharide comprising at least two monosaccharide units, which is exogenously added to the culture medium and/or has been produced by a separate microbial fermentation. In a preferred embodiment the substrate for HMO formation is lactose which is fed to the culture during the fermentation of the genetically engineered cell. Alternative acceptor saccharides can for example be LNT-II, 2′FL, 3FL, 3′SL, 6′SL, LNnT and LNT which can be exogenously added to the culture medium and/or has been produced by a separate microbial fermentation. Preferably, the acceptor saccharide is provided at the end of the batch phase if the fermentation is a fed-batch or continuous fed-batch fermentation.

Culturing or fermenting (used interchangeably herein) in a controlled bioreactor typically comprises (a) a first phase of exponential cell growth in a culture medium ensured by a carbon source, and (b) a second phase of cell growth in a culture medium run under carbon limitation, where the carbon source is added continuously together with the acceptor oligosaccharide, such as lactose, allowing formation of the HMO product in this phase. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter.

In relation to the present disclosure the term “in excess of carbon source” is understood as an amount of carbon source resulting in a growth rate that is between 50% of the maximum growth rate and the maximum growth rate. When a cell growth in excess of a carbon source, the overflow metabolism of the cell is activated, and the cell will start to produce acetate and/or ethanol. In non-limiting examples an excess of carbon source is above 0.05 mM carbon source, such as above 0.75 mM, preferably above 1 mM carbon source when the cell is grown at between 28° C. and 33° C.

One embodiment of the present disclosure is a method for producing a human milk oligosaccharide (HMO) comprising the steps of a) fermenting a genetically modified cell according to the present disclosure in at least 1,000 L cell culture medium comprising an initial amount of carbon source (batch phase); and b) initiating feeding the fermentation with carbon source and acceptor oligosaccharide once the carbon source in step a) is consumed (feeding phase) to produce a fermentation broth comprising the HMO producing microorganism(s) and one or more HMO product(s), wherein the acetate levels in the batch phase of the fermentation is below 5.0 g/L, preferably below 4.0 g/L, preferably below 3.5 g/L, more preferably below 3.0 g/L, most preferably below 2.5 g/L at any timepoint during the batch phase of the fermentation, and wherein the acetate levels in the feeding phase is below 500 mg/L, preferably below 400 mg/L, more preferably below 300 mg/L most preferably below 250 mg/L at any timepoint during the feeding phase of the fermentation. Preferably, the HMO product is retrieved from the fermentation at the end of fermentation.

At the end of culturing/fermentation, the HMO product can be accumulated both in the intra- and the extracellular matrix from which it can be retrieved as described in the section “Retrieving/Harvesting”.

The terms “manufacturing” or “manufacturing scale” or “large-scale production” or “large-scale fermentation”, are used interchangeably and in the meaning of the disclosure defines a fermentation with a minimum volume of 1,000 L, such as 10,000 L, such as 50,000 L, such as 100,000 L, such as 200,000 L, such as 300,000 L culture broth. Usually, a “manufacturing scale” process is defined by being capable of processing large volumes yielding amounts of the HMO product of interest that meet, e.g., in the case of a therapeutic compound or composition, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behavior of an expression system in a lab scale method, such as shake flasks benchtop bioreactors or the deep well format described in the examples of the disclosure, does allow to predict the behavior of that system in the complex environment of a bioreactor.

With regards to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. The carbon source can be selected from the group consisting of glucose, sucrose, fructose, xylose and glycerol. In one or more exemplary embodiments, the culturing media is supplemented with one or more energy and carbon sources selected form the group containing glycerol, sucrose and glucose. Preferably, the carbon source is glucose or sucrose.

In one or more exemplary embodiments, the culturing media contains sucrose as the sole carbon and energy source. In one or more exemplary embodiments, the genetically engineered cell comprises one or more heterologous nucleic acid sequence encoding one or more heterologous polypeptide(s) which enables utilization of sucrose as sole carbon and energy source of said genetically engineered cell, such as a PTS-dependent sucrose utilization system, further comprising the scrYA and scrBR operons as described in WO2015/197082.

After carrying out the method of the present disclosure, the HMO produced can be collected from the cell culture or fermentation broth in a conventional manner.

### Retrieving/Harvesting

The human milk oligosaccharide (HMO) is retrieved from the culture medium and/or the genetically modified cell. In the present context, the term “retrieving” is used interchangeably with the term “harvesting”. Both “retrieving” and “harvesting” in the context relate to collecting the produced HMO(s) from the culture/broth following the termination of fermentation. In one or more exemplary embodiments it may include collecting the HMO(s) included in both the biomass (i.e., the host cells) and cultivation media, i.e., before/without separation of the fermentation broth from the biomass. In other embodiments, the produced HMOs may be collected separately from the biomass and fermentation broth, i.e., after/following the separation of biomass from cultivation media (i.e., fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation broth) include extraction thereof from the biomass (i.e., the production cells).

In particular when for the produced HMOs are not readily exported out of the cell, the HMO can be obtained by perforating the cells to allow the HMO to be released from the cells, without necessarily disrupting the cells entirely. The advantage of this is that proteins, DNA and other molecules still remain in the cells, thereby simplifying the downstream purification of the HMO.

After recovery from fermentation, HMO(s) are available for further processing and purification.

The HMOs can be purified according to the procedures known in the art, e.g., such as described in WO2017/182965 or WO2017/152918, wherein the latter describes purification of sialylated HMOs. The purified HMOs can be used as nutraceuticals, pharmaceuticals, or for any other purpose, e.g., for research.

### Manufactured Product

The term “manufactured product” according to the use of the genetically engineered cell or the nucleic acid construct refer to the one or more HMOs intended as the one or more product HMO(s). The various products are described above.

The manufactured product may be a powder, a composition, a suspension, or a gel comprising one or more HMOs.

### Sequences

The current application contains a sequence listing in text format and electronical format which is hereby incorporated by reference.

An overview of the SEQ ID NOs used in the present application are shown in the table.

Summary of sequences listed in the application

| | | SEQ ID |
|---|---|---|
| Name | Comments | NO |
| gltA | Citrate synthase protein sequence | 1 |
| ppc | Phosphoenolpyruvate carboxylase | 2 |
| | protein sequence | |
| gltA | Citrate synthase DNA coding | 3 |
| | sequence | |
| ppc | Phosphoenolpyruvate carboxylase | 4 |
| | DNA coding sequence | |
| Promoter | Promoter sequences listed | 5-28 |
| sequences | in table 10 | |
| Pcon3_70UTR | Promoter sequence | 29 |
| iclR | isocitrate lyase regulator family | 30 |
| | DNA sequence | |

### Items

Below is a list of preferred items of the present application
1. A genetically modified cell capable of producing one or more Human Milk Oligosaccharides (HMOs), wherein said cell comprises the following modifications:
   a) overexpression of citrate synthase (gltA), and
   b) one or more heterologous nucleic acids encoding one or more glycosyltransferases, and
   c) at least one biosynthetic pathway for making an activated sugar nucleotide capable of serving as glycosyl-donor for the glycosyl transferase(s) of b).
2. The genetically modified cell according to any of items 1, wherein the overexpression of the citrate synthase is achieved by one or more of the following modifications:
   a) placing the native gltA gene under control of a promoter that is stronger than the native promoter, or
   b) inserting a nucleic acid encoding a citrate synthase comprising or consisting of the amino acid sequence of SEQ ID NO: 1 or an amino acid sequence with at least 80% identity to SEQ ID NO: 1 into said cell.
3. The genetically modified cell according to any of items 1 or 2, wherein the cell further comprises at least one of the following modifications
   i. overexpression of phosphoenolpyruvate carboxylase (ppc), and/or
   ii. decreased or total loss of function of the isocitrate lyase regulator (iclR).
4. The genetically modified cell according to item 3, wherein the wherein the overexpression of the phosphoenolpyruvate carboxylase is achieved by one or more of the following modifications:
   a) placing the native ppc gene under control of a promoter that is stronger than the native promoter, or
   b) inserting a nucleic acid encoding a phosphoenolpyruvate carboxylase comprising or consisting of the amino acid sequence of SEQ ID NO: 2 or an amino acid sequence with at least 80% identity to SEQ ID NO: 2 into said cell.
5. The genetically modified cell according to item 3 or 4, wherein the gene encoding the isocitrate lyase regulator (iclR) is deleted or rendered dysfunctional.
6. The genetically modified cell according to any one of the preceding items, wherein the overexpression of the gltA and/or ppc gene(s) is under control of a promoter selected from promotor sequences with a nucleic acid sequence as identified in Table 10, preferably under control of a promoter sequence selected from the group consisting of SEQ ID NO: 17 (PglpF) or SEQ ID NO: 26 (Plac) or SEQ ID NO: 14 (PmglB_UTR70) or SEQ ID NO: 15 (PglpA_70UTR) or SEQ ID NO: 16 (PglpT_70UTR) or SEQ ID NO: 29 (Pcon3_70UTR) or variants of these.
7. The genetically modified cell according to any one of the preceding items, wherein the nucleic acid encoding citrate synthase (gltA) and/or phosphoenolpyruvate carboxylase (ppc), is inserted into the genome of the genetically modified cell.
8. The genetically modified cell according to any one of the preceding items, where the one or more glycosyltransferase(s) is selected from the group of enzymes having the activity of an α-1,2-fucosyltransferase, α-1,3-fucosyltransferase, α-1,3/4-fucosyltransferase, α-1,4-fucosyltransferase α-2,3-sialyltransferase, α-2,6-sialyltransferase, β-1,3-N-acetylglucosaminyltransferase, β-1,6-N-acetylglucosaminyltransferase, β-1,3-galactosyltransferase and β-1,4-galactosyltransferase.
9. The genetically modified cell according to item 8, where the one or more glycosyltransferase(s) is selected from α-1,2-fucosyltransferase, α-1,3-fucosyltransferase, and/or α-1,3/4-fucosyltransferase of table 4, 5 or 6.
10. The genetically modified cell according to any one of the preceding items, wherein the glycosyltransferase is a fucosyltransferase and at least one of the genes in the biosynthetic pathway necessary for the de novo synthesis of GDP-fucose is overexpressed.
11. The genetically modified cell according to item 10, wherein the genes manB, manC, gmd and wcaG of the de novo GDP-fucose pathway are all overexpressed.
12. The genetically modified cell according to item 10 or 11, wherein manA is overexpressed.
13. The genetically modified cell according to any one of items 1 to 8 or 10 to 12, wherein the one or more HMO(s) is selected from the group consisting of: LNT, LNnT, LNH, LNnH, pLNH, pLNnH, 2′FL, 3FL, DFL, LNFP-1, LNFP-II, LNFP-III, LNFP-V, LNFP-VI, LNDFH-1, LNDFH-II, LNDFH-III, F-pLNH, pLNnH, FLSTa, FLSTb, FLSTc, FLSTd, FSL, 3′SL, 6′SL, LSTa, LSTb, LSTc, LSTd, DSLNT, SLNH and SLNH-II.
14. The genetically modified cell according any one of the preceding items, wherein the one or more HMOs are fucosylated, preferably selected from the group consisting of: 2′FL, 3FL, DFL, LNFP-1, LNFP-II, LNFP-III, LNFP-V, LNFP-VI LNDFH-1, LNDFH-II and LNDFH-III.
15. The genetically modified cell according to any one of the preceding items, wherein the cell further comprises a nucleic acid sequence encoding a transporter protein capable of exporting the produced HMO into the extracellular medium.
16. The genetically modified cell according to any of the preceding items, wherein said modified cell is a microorganism.
17. The genetically modified cell according to any of the preceding items, wherein said modified cell is a bacterium or a fungus.
18. The genetically modified cell according to item 16 or 17, wherein said fungus is a yeast, preferably selected from the group consisting of Yarrowia lipolytica, Pichia pastoris, and Saccharomyces cerevisiae.
19. The genetically modified cell according to item 16 or 17, wherein said fungus is a filamentous fungous, preferably selected from the genera Aspergillus sp., Fusarium sp. or Trichoderma sp.
20. The genetically modified cell according to item 16 or 17, wherein said bacterium is selected from the group consisting of Escherichia sp., Bacillus sp., Lactobacillus sp and Corynebacterium sp. Campylobacter sp.
21. The genetically modified cell according to item 20, wherein said bacterium is an Escherichia coli K12.
22. A method for producing a human milk oligosaccharide (HMO) comprising the steps of:
   a) providing a genetically modified cell according to any one of items 1 to 21, and
   b) culturing the cell according to (a) in a suitable cell culture medium to produce said HMO.
23. The method according to item 22, wherein the HMO is recovered from the cultivation broth and/or the biomass.
24. The method according to item 22 or 23, wherein the method comprises cultivating the genetically engineered cell in a culture medium which contains one or more carbon sources selected from the group consisting of glucose, sucrose, fructose, xylose and glycerol.
25. The method according to item 24, wherein the carbon source is glucose or sucrose.
26. The method according to any one of items 22 to 25, wherein the culture medium in which the cultivation is conducted contains lactose.
27. The method according to any one of items 22 to 26, wherein the cultivation is a fed batch fermentation or a feed and bleed fermentation, where the carbon sources is continuously feed to the fermentation broth.
28. The method according to item 27, where the lactose is absent in the batch phase and added to the culture during the feeding phase.
29. The method according to any of items 22 to 28, wherein the genetically modified cell genetically modified cell overexpress citrate synthase (gltA) and phosphoenolpyruvate carboxylase (ppc) and the acetic acid formation in cultures grown in excess of carbon source is at least 30% lower as compared to a method where the genetically modified cell does not overexpress citrate synthase (gltA) and phosphoenolpyruvate carboxylase (ppc).
30. The method according to item any of items 22 to 27, wherein the genetically modified cell genetically modified cell overexpress citrate synthase (gltA) and isocitrate lyase regulator (iclR) expression is abolished and the acetic acid formation in cultures grown in excess of carbon source is at least 30% lower as compared to a method where the genetically modified cell does not overexpress citrate synthase (gltA) and isocitrate lyase regulator (iclR) expression is abolished.
31. The method according to item any of items 22 to 28, wherein the genetically modified cell according to items 1 to 18 produce less ethanol as compared to a cell not containing the item modifications.
32. The method according to item any of items 22 to 31, wherein the one or more HMO(s) is selected from the group consisting of: LNT, LNnT, LNH, LNnH, pLNH, pLNnH, 2′FL, 3FL, DFL, LNFP-I, LNFP-II, LNFP-III, LNFP-V, LNFP-VI, LNDFH-I, LNDFH-II, LNDFH-III, F-pLNH, pLNnH, FLSTa, FLSTb, FLSTc, FLSTd, FSL, 3′SL, 6′SL, LSTa, LSTb, LSTc, LSTd, DSLNT, SLNH and SLNH-II.

### EXAMPLES

### Methods

Unless stated otherwise, standard techniques, vectors, control sequence elements, and other expression system elements known in the field of molecular biology are used for nucleic acid manipulation, transformation, and expression. Such standard techniques, vectors, and elements can be found, e.g., in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972) (Cold spring Harbor Laboratory Press, NY)

The embodiments described below are selected to illustrate the invention and are not limiting the invention in any way.

### Strains

The strains (genetically engineered cells) constructed in the present application were based on Escherichia coli K-12 DH1 with the genotype: F⁻, k, gyrA96, recA1, relA1, endA1, thi-1, hsdR17, supE44. Additional modifications were made to the E. coli K-12 DH1 strain to generate the MDO strain with the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene.

Methods of inserting or deleting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186: 2673-81 and Warming et al 2005 Nucleic Acids Res. 33(4): e36) with specific selection marker genes and screening methods.

The MDO strain was further engineered to generate a 2′FL producing strain by chromosomally integrating two copies of the alpha-1,2-fucosyltransferase futC from Helicobacter pylori 26695 (homologous to NCBI Accession nr. WP_080473865.1 with two additional amino acids (LG) at C-terminus) under control of the PglpF promoter and an additional copy of the colonic acid operon (gmd-wcaG-wcaH-wcal-manC-manB) under control of the PglpF promter.

The genotypes of the background strain (MDO), 2′FL strain and the stains used in the examples are shown in Table 11 below.

TABLE 11

| Strain | | |
|---|---|---|
| ID | Modification | Genotype |
| MDO | | F- λ- ΔendA1 ΔrecA1 ΔrelA1 |
| | | ΔgyrA96 Δthi-1 glnV44 hsdR17(rK-mK-) |
| | | ΔlacZ wcaF::Plac ΔnanKETA |
| | | ΔlacA ΔmelA ΔwcaJ ΔmdoH |
| Control | 2′FL | MDO, 2x FutC-PglpF, 1xCA |
| MP1 | gltA | 2′FL, gltA-PglpF |
| MP2 | ΔiclR | 2′FL, ΔiclR |
| MP3 | gltA, ΔiclR | 2′FL, gltA-PglpF, ΔiclR |
| MP4 | ppc | 2′FL, ppc-PglpF |
| MP5 | gltA, ppc | 2′FL, gltA-PglpF, ppc-PglpF |

### Deep Well Assay

The strains were screened in 96 deep well plates using a 3-day protocol for 2′FL and a 2-day protocol with high glucose for acetate assessment and growth rate assessment. During the first 24 hours, fresh precultures were grown to high densities and subsequently transferred to a medium that allowed for product formation or induced acetate formation. More specifically, fresh precultures were prepared using a basal minimal medium supplemented with magnesium sulphate, thiamine and glucose The pH was adjusted to pH 7.0 with NaOH. The precultures were incubated for 24 hours at 34° C. and 1000 rpm shaking.

For 2′FL production an aliquot of the pre-culture was transferred to a new deep well plate with basal minimal medium (BMM, pH 7.5) to start the main culture. The new BMM was supplemented with thiamine and magnesium sulphate, 0.01% of glucose, 0.5% lactose, 3.75% of sucrose, thiamine and 0.1 g/L sucrose hydrolase (Sigma-Aldrich). The 2′FL main cultures were incubated for 48 hours at 30° C. and 1000 rpm shaking.

For acetate production, an aliquot of the of the pre-culture was transferred into new deep-well plates, with BMM medium (pH 7.5) with thiamine and magnesium sulphate and 2.5% of glucose. The acetate main cultures were incubated at 30° C., 1000 prm for 24 hours.

After incubation of the main cultures were boiled and centrifuged. Samples were analysed by HPLC and Acetic Acid Assay Kit (Megazyme).

For growth measurement, an aliquot of the pre-culture was transferred into microtiter plates, with BMM medium (pH 7.5) with thiamine and magnesium sulphate and 2.5% of glucose. The plates were incubated at 30° C., 840 rpm for 24 hours in Varioskan. Absorbance at 600 nm was measured every 30 minutes.

### Carbon-Limited Feed-Batch Fermentation

Precultures for the fed-batch fermentations were prepared in two steps. Firstly, strains from cryostocks were inoculated into 10 mL of glucose minimal medium (GMM) (10 g/L of NH₄H₂PO₄, 5 g/L of KH₂PO₄, 1 g/L of citric acid, 2.35 g/L of NaOH, 1.65 g/L of KOH, 5 g/L of K₂SO₄ and 5 mL/L of trace mineral solution) with 10 g/L of glucose, 1 g/L of MgSO₄ and 4 μg/mL of thiamine in shake flasks. The pH was adjusted to pH 7.0. The cultures were cultivated at 33° C., 200 rpm, overnight. Then, overnight cultures were inoculated into 50 mL of GMM medium with 10 g/L of glucose, 1 g/L of MgSO₄ and 4 μg/mL of thiamine in shake flasks to reach OD of 0.25. After growing the precultures to an OD of 2-3.5, they were used to inoculate fed-batch fermentations with an inoculation ratio of 2%.

High cell density, glucose limited fed-batch fermentations were carried out in DASbox® Mini Bioreactor System (Eppendorf) with 100 mL of initial volume of defined glucose minimal medium (GMM) mineral culture medium consisting of (NH₄)H₂PO₄, KH₂PO₄ NaOH, KOH, K₂SO₄, citric acid, trace mineral solution, MgSO₄, thiamine and glucose. The pH was adjusted to pH 6.8.

All main cultures started with a batch phase at 33° C. with 25 g/L of glucose. A feed start was triggered by an increase in pH indicating the cells had consumed the initial glucose. Dissolved oxygen was kept at 23% by stirring (minimum 700 rpm) and airflow (6 L/L/min). The pH level was maintained at 6.8 by 14% NH₄OH. A constant feed profile in a carbon-limited manner was used and the feeding medium contained glucose, lactose and all the required nutrients. The fermentation was stopped after 75 hours of feeding phase. 2′FL, bio-wet mass (BWM) and OD (600 nm) were measured from the fermentation broth.

Stress Testing with Large Glucose Pulse During Fed-Batch Process

In the carbon-limited feed-batch fermentation process described above a glucose pulse was added approximately 45-48 hours after the feed started by a fast injection using high concentrated solution and a sterile syringe to obtain 10 g/L (55 mM) of glucose concentration in the fermenter. Samples for acetate and glucose were taken before the pulse, right after the pulse and further at 10, 30, 50, 70 and 90 minutes after the pulse. Aeration and stirring were set to maximum at the glucose pulse addition, thus, dissolved oxygen levels did not reach 0% or only for a limited time. Acetic acid concentrations were quantified from the supernatants by NMR where 2′FL was measured by HPLC.

### Example 1—Acetate Reduction in Deep Well Assay

In the present example the effect of mutations affecting the TCA cycle was investigated both in terms of reduction of acetate, growth rate under high glucose conditions and the ability to maintain 2′FL expression under normal conditions.

The strain indicated in Table 11 were cultivated in the deep well assay described in the Method section above. The results are shown in Table 12 below.

TABLE 12
Strain performances measured in growth rate (μmax), acetate yield and 2′FL titer.
The 2′FL formation is presented relative to the control and as the average of three
individual cultures. Standard deviation of three individual cultures is shown n = 3.

| | | μₘₐₓ | Acetate | Y_{acetate/glucose} | 2′FL |
|---|---|---|---|---|---|
| Genotype | Strain ID | (h⁻¹) | [g/L] | (g/g) | (% of control) |
| | Control | 0.21 ± 0.01 | 3.4 ± 0.13 | 0.17 ± 0.01 | 100.00 ± 4.67 |
| OE gltA | MP1 | 0.20 ± 0.01 | 2.9 ± 0.08 | 0.15 ± 0.01 | 95.40 ± 1.45 |
| ΔiclR | MP2 | 0.23 ± 0.00 | 3.2 ± 0.04 | 0.17 ± 0.00 | 128.70 ± 4.09 |
| OE gltA ΔiclR | MP3 | 0.20 ± 0.01 | 1.0 ± 0.07 | 0.05 ± 0.00 | 123.30 ± 4.71 |
| OE ppc | MP4 | 0.18 ± 0.01 | 2.6 ± 0.05 | 0.13 ± 0.00 | 94.36 ± 1.11 |
| OE gltA + ppc | MP5 | 0.18 ± 0.00 | 1.8 ± 0.02 | 0.09 ± 0.00 | 93.42 ± 3.03 |

OE = overexpression

Single overexpression of gltA (MP1) or ppc (MP4) decreased acetate production by 13.5% and 25%, respectively. Whereas overexpression of both gltA and ppc genes (MP5) led to the acetate reduction by 49%. The combination of iclR deletion and gltA overexpression (MP3) resulted in 73% of acetate yield reduction. However, iclR deletion itself (MP2) only decreased the acetate production by 5%, indicating a synergistic effect of the double mutation.

Production of 2′FL was not significantly affected in any of the strain, although the strains with iclR deletions (MP2 and MP3) appear to show a positive effect in 2′FL production. Growth rates were not affected significantly either.

### Example 2-2′FL and Biomass Production in Fermentation of Strains with gltA Overexpression and or iclR Deletion

Based on the results from example 1 it was decided to test the stains with gltA overexpression and/or iclR deletion in a bioreactor setup.

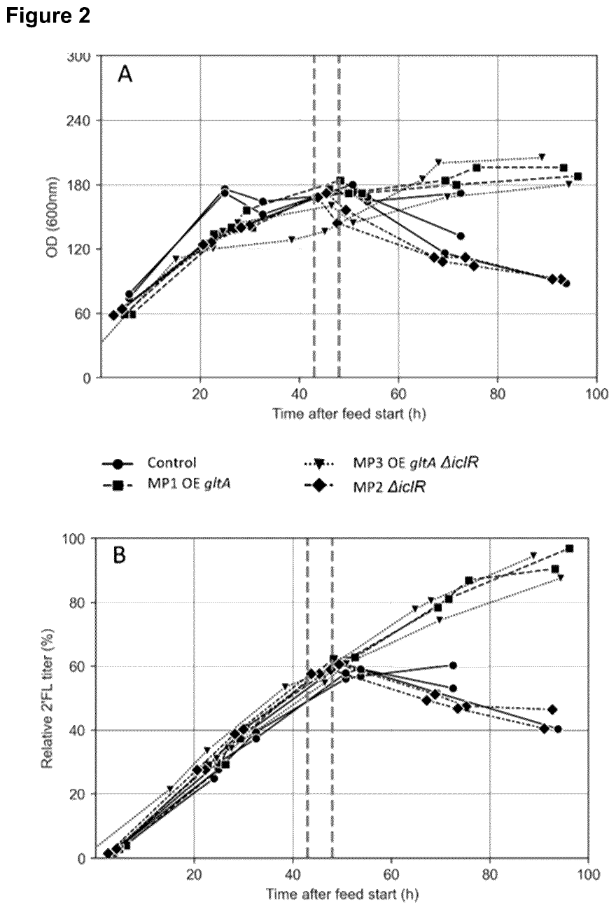

The control strain and MP1, MP2 and MP3 strains were cultivated according to the carbon-limited fermentation protocol in the Method section above. MP1, MP2 and MP3 were fermented as duplicates and the control was fermented in triplicates. The results are shown in Table 13 below and are provided as relative values in % of control at 40 hours after feed start. The values are relative to the average calculated from triplicate fermentations with the control strain or duplicates of the test strains and are given in percentage. FIGS. 2A and 2B also represent the result of this experiment from the time between 0 and 40 h.

TABLE 13
Comparison of 2′FL yields (g 2′FL/g glucose consumed) and biomass
yield (g biomass/g glucose consumed) in fed-batch fermentation.

| | | 2′FL % of control | | biomass | |
|---|---|---|---|---|---|
| Genotype | Strain ID | Average | Std | Average | Std |
| | Control | 100 | 0.03 | 100 | 0.04 |
| OE gltA | MP1 | 107.8 | 0.6 | 117.3 | 4.5 |
| ΔiclR | MP2 | 106.5 | 2.8 | 111.0 | 3.7 |
| OE gltA ΔiclR | MP3 | 105.3 | 4.8 | 123.2 | 1.8 |

Performance of the unstressed mutants were compared 40 hours after the feeding was initiated (Table 13). 2′FL yields were in general similar or slightly higher compared to the control strain. Single overexpression of gltA or deletion of iclR (MP1 and MP2) increased the 2′FL yield on glucose by 7-8% and the double mutant (MP3) had a 5% increase.

### Example 3: Stress Testing with Large Glucose Pulse During Fed-Batch Process

In the current example, the fermentations of example 2 were subjected to a glucose stress test using conditions that resemble large scale fermentations.

After around 40 hours of feeding the fermentations in example 2 were subjected to a large glucose pulse (see stress testing Method above) to mimic high glucose zones, arising in industrial large-scale fermenters in order to stress the strains. Acetate formation was monitored for 90 minutes after the glucose pulse was added. 2′FL and biomass was monitored regularly before (example 2) and after the pulse as well. The results relating to acetate formation are shown in Table 14 and FIG. 1 and the 2′FL and biomass formation is shown FIG. 2.

TABLE 14
Average acetate formation after the glucose pulse was added to the fermentation.

| Strain | | | MP1 | | MP2 | | MP3 | |
|---|---|---|---|---|---|---|---|---|
| genotype | control | | OE gltA | | ΔiclR | | OE gltA ΔiclR | |
| Time | Acetate | | Acetate | | Acetate | | Acetate | |
| min after pulse | g/L | std | g/L | std | g/L | std | g/L | std |
| −5.00 | 0.08 | 0.03 | 0.01 | 0.01 | 0.08 | 0.00 | 0.02 | 0.04 |
| 0.00 | 0.07 | 0.01 | 0.02 | 0.00 | 0.07 | 0.01 | 0.01 | 0.01 |
| 10.00 | 0.24 | 0.09 | 0.10 | 0.02 | 0.17 | 0.03 | 0.05 | 0.02 |
| 30.00 | 0.59 | 0.11 | 0.27 | 0.02 | 0.49 | 0.12 | 0.14 | 0.01 |
| 50.00 | 1.02 | 0.08 | 0.71 | 0.19 | 0.80 | 0.25 | 0.29 | 0.01 |
| 70.00 | 1.88 | 0.04 | 1.35 | 0.36 | 1.62 | 0.59 | 0.64 | 0.22 |
| 90.00 | 2.87 | 0.40 | 1.94 | 0.30 | 2.55 | 0.73 | 1.02 | 0.06 |

All cultures accumulated acetate after the glucose pulse. The two gltA overexpressing strains MP1 and MP3 produced 33% and 64% less acetate than the control strain after 90 minutes, with an average acetate concentrations of 1.9 g/L and 1 g/L, respectively. The AiclR mutant (MP2) accumulated close to an average of 2.6 g/L of acetate after 90 minutes, which is only 10% less than for the control strain which accumulated 2.9 g/L acetate.

In FIG. 2A, it can be seen that prior to the glucose pulse (example 2) the growth of the strains is fairly similar with a slightly faster biomass development in the control strain. After the glucose pulse (indicated by the vertical grey doted lines) the growth of the control strain and the AiclR strain (MP2), declined rapidly, indicating that these strains do not tolerate the glucose pulse, which concur with the immediate acetate increase observed for these strains in Table 14 and FIG. 1. The high level of acetate clearly impairs the cell growth and most likely kill the cells in the fermentation.

FIG. 2B shows the 2′FL formation (g 2′FL/g glucose) relative to the maximal 2′FL production (MP1 at 97 h) in percentage. As described in Example 2 the 2′FL formation is fairly similar for the first 40 hours with a slight increase in 2′FL formation in all the mutated strains (see Table 13). However, as with the biomass reduction, the 2′FL formation also decreases rapidly for the control strain and the ΔiclR strain (MP2), whereas the 2′FL formation for the cells overexpressing gltA appears to be unaffected by the glucose pulse.

1. A genetically modified cell capable of producing one or more Human Milk Oligosaccharides (HMOs), wherein the cell comprises the following modifications:
a) overexpression of citrate synthase (gltA),
b) one or more heterologous nucleic acids encoding one or more glycosyltransferases, and
c) at least one biosynthetic pathway for making an activated sugar nucleotide capable of serving as glycosyl-donor for the one or more glycosyl-transferase(s).

2. The genetically modified cell according to claim 1, wherein the overexpression of the citrate synthase is achieved by one or more of the following modifications:
d) placing the native gltA gene under control of a promoter that is stronger than the native promoter, and/or
e) inserting a nucleic acid encoding a citrate synthase comprising amino acid sequence with at least 80% identity to SEQ ID NO: 1 into the cell.

3. The genetically modified cell according to claim 1, wherein the cell further comprises one or more of the following modifications
i. overexpression of phosphoenolpyruvate carboxylase (ppc), and/or
ii. decreased or total loss of function of the isocitrate lyase regulator (iclR).

4. The genetically modified cell according to claim 3, wherein the wherein the overexpression of the phosphoenolpyruvate carboxylase is achieved by one or more of the following modifications:
f) placing the native ppc gene under control of a promoter that is stronger than the native promoter, and/or
g) inserting a nucleic acid encoding a phosphoenolpyruvate carboxylase comprising or consisting of the amino acid sequence of SEQ ID NO: 2 or an amino acid sequence with at least 80% identity to SEQ ID NO: 2 into the cell.

5. The genetically modified cell according to claim 3, wherein the gene encoding the isocitrate lyase regulator (iclR) is deleted or rendered dysfunctional.

6. The genetically modified cell according to claim 1, wherein the overexpression of the gltA and/or ppc gene(s) is under control of a promoter selected from promotor sequences with a nucleic acid sequence selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 and SEQ ID NO: 29.

7. (canceled)

8. The genetically modified cell according to claim 1, where the one or more glycosyltransferase(s) is selected from the group of enzymes having the activity of an α-1,2-fucosyltransferase, α-1,3-fucosyltransferase, α-1,3/4-fucosyltransferase, α-1,4-fucosyltransferase α-2,3-sialyltransferase, α-2,6-sialyltransferase, β-1,3-N-acetylglucosaminyltransferase, β-1,6-N-acetylglucosaminyltransferase, β-1,3-galactosyltransferase and β-1,4-galactosyltransferase.

9. The genetically modified cell according to claim 1, wherein the one or more glycosyltransferase(s) is a fucosyltransferase and at least one of the genes in the biosynthetic pathway necessary for the de novo synthesis of GDP-fucose is overexpressed.

10. The genetically modified cell according to claim 1, wherein the one or more HMO(s) is selected from the group consisting of: LNT, LNnT, LNH, LNnH, pLNH, pLNnH, 2′FL, 3FL, DFL, LNFP-I, LNFP-II, LNFP-III, LNFP-V, LNFP-VI, LNDFH-I, LNDFH-II, LNDFH-III, F-pLNH, pLNnH, FLSTa, FLSTb, FLSTc, FLSTd, FSL, 3′SL, 6′SL, LSTa, LSTb, LSTc, LSTd, DSLNT, SLNH and SLNH-II.

11. The genetically modified cell according claim 1, wherein the one or more HMOs are fucosylated.

12. The genetically modified cell according to claim 1, wherein the cell further comprises a nucleic acid sequence encoding a transporter protein capable of exporting the produced HMO into the extracellular medium.

13-16. (canceled)

17. The genetically modified cell according to claim 1, wherein the modified cell is a microorganism selected from the group consisting of Escherichia sp., Bacillus sp., Lactobacillus sp., Corynebacterium sp., Campylobacter sp., Yarrowia lipolytica, Pichia pastoris, Saccharomyces cerevisiae, Aspergillus sp., Fusarium sp. and Trichoderma sp.

18. A method for producing α-one or more human milk oligosaccharide (HMOs) comprising the steps of:
h) providing a genetically modified cell according to claim 1, and
i) culturing the cell in a suitable cell culture medium to produce the one or more HMOs.

19. The method according to claim 18, wherein the HMO is recovered from a cultivation broth and/or biomass.

20. The method according to claim 18, wherein the culture medium contains one or more carbon sources selected from the group consisting of glucose, sucrose, fructose, xylose and glycerol.

21. The method according to claim 20, wherein the carbon source is glucose or sucrose.

22. The method according to claim 18, wherein the culture medium contains lactose.

23-24. (canceled)

25. The method according to claim 18, wherein the genetically modified cell genetically modified cell overexpress citrate synthase (gltA) and phosphoenolpyruvate carboxylase (ppc) and the acetic acid formation in cultures grown in excess of carbon source is at least 30% lower as compared to a method where the genetically modified cell does not overexpress citrate synthase (gltA) and phosphoenolpyruvate carboxylase (ppc).

26. The method according to claim 18, wherein the genetically modified cell genetically modified cell overexpress citrate synthase (gltA) and isocitrate lyase regulator (iclR) expression is abolished and the acetic acid formation in cultures grown in excess of carbon source is at least 30% lower as compared to a method where the genetically modified cell does not overexpress citrate synthase (gltA) and isocitrate lyase regulator (iclR) expression is abolished.

27. (canceled)

28. The method according to claim 18, wherein the one or more HMO(s) is selected from the group consisting of LNT, LNnT, LNH, LNnH, pLNH, pLNnH, 2′FL, 3FL, DFL, LNFP-I, LNFP-II, LNFP-III, LNFP-V, LNFP-VI, LNDFH-I, LNDFH-II, LNDFH-III, F-pLNH, pLNnH, FLSTa, FLSTb, FLSTc, FLSTd, FSL, 3′SL, 6′SL, LSTa, LSTb, LSTc, LSTd, DSLNT, SLNH and SLNH-II.
